(19) United States
(12) Patent Application Publication
(10) Pub. No.: US 2008/0254035 A1
(43) Pub. Date: Oct. 16, 2008

(54) Lipocalin Protein

(75) Inventors: Melanie Yorke-Smith, Confignon (CH); Christine Power, Thoiry (FR); Ursula Boschert, Troinex (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Ares Trading S.A., Aubonne (CH)

(21) Appl. No.: 11/817,296

(22) PCT Filed: Mar. 8, 2006
(86) PCT No.: PCT/GB06/00820

§ 371 Date: Feb. 19, 2008

(30) Foreign Application Priority Data
Mar. 8, 2005 (GB) 0504767.5

(51) Int. Cl. A61K 39/395; C07K 14/00; C12N 15/11; C12N 15/00; C12N 5/06; C07K 16/18; A61K 39/00; C40B 30/04; A01K 67/027; A61P 43/00; G01N 33/566; C12Q 1/68; A61K 38/43; C12N 9/00; A61K 38/00; A61K 31/70; A61K 35/00

(52) U.S. Cl. 424/139.1; 530/350; 536/23.1; 435/320.1; 435/325; 530/387.9; 435/183; 514/12; 514/44; 424/93.2; 424/94.1; 424/185.1; 435/6; 506/17; 436/501; 800/13

(57) ABSTRACT

The invention is based on the discovery that the human protein referred to herein as INSP181 protein is a lipocalin.

This invention relates to a novel protein (termed INSP181), and derivatives thereof, herein identified as a lipocalin and to the use of this protein, and nucleic acid sequences from the encoding gene, in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Lipocalins are small secreted proteins that are believed to be involved in the transport of small, hydrophobic molecules. Lipocalins are characterized by a multi-domain structure comprising a ligand binding domain that is typically involved in binding small, hydrophobic molecules and a conserved cell-surface receptor-binding domain that is typically involved in binding some putative cell-surface receptor that may be common to more than one lipocalin and open end of the fold structure that forms a macromolecular complex, perhaps involving the cell-surface receptor.

In spite of large diversity at the sequence level, lipocalins are structural homologues: a single eight-stranded antiparallel β-barrel with an attached α-helix forms the distinct “lipocalin scaffold”. One end of the barrel is opened to the solvent and contains a ligand binding site. A set of four loops connecting consecutive strands confer specificity for ligand binding.

The most related members of the lipocalin family share three characteristic conserved sequence motifs. Members of this group include: retinol-binding protein; purpurin; retinoic acid-binding protein; alpha-2-microglobin; major urinary protein; bilin-binding protein; alpha-crustacyanin; pregnancy protein 14; beta-lactoglobin; neutrophil lipocalin and choroid plexus protein. Outlier lipocalins are classified as such because they have 2 or less sequence motifs conserved and these proteins include: odorant-binding protein, von Ebner's gland protein, probasin and aphrodisin.

The identification of lipocalins is therefore of extreme importance in increasing the understanding of the underlying pathways that lead to certain disease states and associated disease states, mentioned below, and in developing more effective gene and/or drug therapies to treat these disorders.

### THE INVENTION

The invention is based on the discovery that the human protein referred to herein as the INSP181 protein is a lipocalin.

The invention is based on the finding that polypeptides of the present invention upregulate T helper cell 2 (Th2) cytokines, more specifically interleukin-10 (IL-10), interleukin-4 (IL-4) and interleukin-5 (IL-5). The INSP181 polypeptide was tested for its effect on cytokine secretion by Human Peripheral Blood Mononuclear Cells (PBMC) stimulated with the mitogen, concanavalin A (ConA). It was found that this polypeptide stimulates IL-10, IL-4 and IL-5 secretion from ConA-stimulated human PBMC when tested at a 1/10 dilution (46.2 ug in the assay). No effect was seen on the levels of IFN-γ, TNF-α, or IL-2.

In addition, it has been surprisingly found that the polypeptides of the present invention show unexpected restricted expression in skin biopsy samples and particularly in psoriasis skin biopsies. In brief, primers specific for INSP181 were tested on a panel of approximately 100 normal and diseased human tissue samples, primary cells and cell lines in addition to 44 inflammatory bowel disease colon and ileum biopsies and 39 psoriasis biopsies from an IL-18 bind protein (IL18BP) clinical trial. Results are shown in tables 3 and 4 and represented graphically in FIGS. 12 and 13. INSP181 expression was surprisingly only detected at low level in skin (0.16% of GAPDH) (Table 5, FIG. 12) and in skin biopsies from psoriasis patients (19/39 samples positive) (table 4, FIG. 13). A second primer pair specific for exons 4/6 (forward primer in exon 4 and reverse primer in exon 6) confirmed the skin specificity.

In a first aspect, the invention provides a polypeptide, which polypeptide:
(i) comprises or consists of the amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO: 66 or SEQ ID NO: 70;
(ii) is a fragment thereof which is a lipocalin or which has an antigenic determinant in common with one or more of the polypeptides of (i); or
(iii) is a functional equivalent of (i) or (ii).

Preferably, the polypeptide according to this first aspect of the invention consists or comprises the amino acid sequence as recited in SEQ ID NO:18 or SEQ ID NO:24, is a fragment thereof which functions as a lipocalin, or has an antigenic determinant in common with such a polypeptide; or is a functional equivalent of such a polypeptide.

The polypeptide having the sequence recited in SEQ ID NO:2 is referred to hereafter as “the INSP181 exon 1 polypeptide”. The polypeptide having the sequence recited in SEQ ID NO:4 is referred to hereafter as “the INSP181 exon 2 polypeptide”. The polypeptide having the sequence recited in SEQ ID NO:6 is referred to hereafter as “the INSP181 exon 3 polypeptide”. The polypeptide having the sequence recited in SEQ ID NO:8 is referred to hereafter as “the INSP181-SV1 exon 3 polypeptide”. The polypeptide having the sequence recited in SEQ ID NO:10 is referred to hereafter as “the INSP181-SV1 exon 4 polypeptide”. The polypeptide having the sequence recited in SEQ ID NO:12 is referred to hereafter as “the INSP181 exon 4 polypeptide”. The polypeptide having the sequence recited in SEQ ID NO:14 is referred to hereafter as “the INSP181 exon 5 polypeptide”. The polypeptide having the sequence recited in SEQ ID NO:16 is referred to hereafter as “the INSP181 exon 6 polypeptide”.

SEQ ID NO:18 is produced by combining SEQ ID NOs: 2, 4, 6, 12, 14 and 16. The polypeptide having the sequence recited in SEQ ID NO:18 is referred to hereafter as “the INSP181 polypeptide”.

SEQ ID NO:24 is produced by combining SEQ ID NOs: 2, 4, 8, 10, 14 and 16. The polypeptide having the sequence recited in SEQ ID NO:24 is referred to hereafter as “the INSP181-SV1 polypeptide”. The INSP181-SV1 protein contains a 25 amino acid insertion at the start of exon 4.

A second INSP181-SV1 clone (INSP181-SV1-polymorph) was identified from a pool containing cDNA derived from salivery gland, adrenal gland, eye, and the Stratagene universal reference RNA template, which contained the INSP181-SV1 cDNA. This clone contains the nucleotide substitutions A275C and G340A which lead to amino acid substitutions N92T and G114S. These are postulated to be polymorphisms in the INSP181 sequence.

The polypeptide having the sequence recited in SEQ ID NO:36 is referred to hereafter as “the INSP181 exon 3 N92T polymorph polypeptide” and comprises the amino acid substitution N92T. The polypeptide having the sequence recited in SEQ ID NO:38 is referred to hereafter as “the INSP181-SV1 exon 3 N92T polymorph polypeptide” and comprises the amino acid substitution N92T.

The polypeptide having the sequence recited in SEQ ID NO:40 is referred to hereafter as “the INSP181N92T polymorph polypeptide”. SEQ ID NO:40 is produced by combining SEQ ID NOs: 2, 4, 36, 12, 14 and 16.

The polypeptide having the sequence recited in SEQ ID NO:44 is referred to hereafter as “the INSP181-SV1 N92T polymorph polypeptide”. SEQ ID NO:44 is produced by combining SEQ ID NOs: 2, 4, 38, 10, 14 and 16.

The polypeptide having the sequence recited in SEQ ID NO:56 is referred to hereafter as “the INSP181-SV1 exon 4 G114S polymorph polypeptide” and comprises the amino acid substitution G114S.

The polypeptide having the sequence recited in SEQ ID NO:58 is referred to hereafter as “the INSP181-SV1 G114S polymorph polypeptide”. SEQ ID NO:58 is produced by combining SEQ ID NOs: 2, 4, 8, 56, 14 and 16.

Although the Applicant does not wish to be bound by this theory, it is postulated that the INSP181 polypeptide, the INSP181-SV1 polypeptide, the INSP181 N92T polymorph polypeptide, the INSP181-SV1 N92T polymorph polypeptide, and the INSP181-SV1 G114S polymorph polypeptide may comprise a signal peptide at the N-terminus that is 20 amino acids in length.

Exon 1 of the INSP181 polypeptide and the INSP181-SV1 polypeptide without this postulated signal sequence is recited in SEQ ID NO:20 and is referred to hereafter as “the INSP181 mature exon 1 polypeptide”. The INSP181 polypeptide sequence without this postulated signal sequence is recited in SEQ ID NO:22 and is referred to hereafter as “the INSP181 mature polypeptide”. The INSP181-SV1 polypeptide sequence without this postulated signal sequence is recited in SEQ ID NO:26 and is referred to hereafter as “the INSP181-SV1 mature polypeptide”. The INSP181 N92T polymorph polypeptide sequence without this postulated signal sequence is recited in SEQ ID NO:42 and is referred to hereafter as “the INSP181 N92T polymorph mature polypeptide”. The INSP181-SV1-N92T polymorph polypeptide sequence without this postulated signal sequence is recited in SEQ ID NO:46 and is referred to hereafter as “the INSP181-SV1-N92T polymorph mature polypeptide”. The INSP181-SV1-G114S polymorph polypeptide sequence without this postulated signal sequence is recited in SEQ ID NO:60 and is referred to hereafter as “the INSP181-SV1-N92T polymorph mature polypeptide”.

The polypeptide having the sequence recited in SEQ ID NO:66 is referred to hereafter as “the lipocalin domain INSP181 polypeptide” and comprises the lipocalin domain. The polypeptide having the sequence recited in SEQ ID NO:70 is referred to hereafter as “the lipocalin domain INSP181-SV1 polypeptide” and comprises the lipocalin domain of the splice variant.

The polypeptides of the first aspect of the invention may further comprise a histidine tag. Preferably the histidine tag is found at the C-terminal of the polypeptide. Preferably the histidine tag comprises 1-10 histidine residues (e.g. 1, 2, 3, 4, 5, 6, 7, 8, 9 or 10 residues). More preferably, the histidine tag comprises 6 histidine residues. Preferred polypeptides are therefore those comprising the sequence recited in SEQ ID NO:28, SEQ ID NO:30, SEQ ID NO:32 SEQ ID NO:34, SEQ ID NO:48, SEQ ID NO:50, SEQ ID NO: 52, SEQ ID NO:54, SEQ ID NO:62, SEQ ID NO:68, and/or SEQ ID NO:72.

The polypeptide having the sequence recited in SEQ ID NO:28 is hereafter referred to as “the his tag INSP181 polypeptide”. The polypeptide having the sequence recited in SEQ ID NO:30 is hereafter referred to as “the mature his tag INSP181 polypeptide”. The polypeptide having the sequence recited in SEQ ID NO:32 is hereafter referred to as “the his tag INSP181-SV1 polypeptide”. The polypeptide having the sequence recited in SEQ ID NO:34 is hereafter referred to as “the mature his tag INSP181-SV1 polypeptide”. The polypeptide having the sequence recited in SEQ ID NO:48 is hereafter referred to as “the his tag INSP181 N92T polymorph polypeptide”. The polypeptide having the sequence recited in SEQ ID NO:50 is hereafter referred to as “the mature his tag INSP181 N92T polymorph polypeptide”. The polypeptide having the sequence recited in SEQ ID NO:52 is hereafter referred to as “the his tag INSP181-SV1 N92T polpmorph polypeptide”. The polypeptide having the sequence recited in SEQ ID NO:54 is hereafter referred to as “the mature his tag INSP181-SV1 N92T polymorph polypeptide”. The polypeptide having the sequence recited in SEQ ID NO:62 is hereafter referred to as “the his tag INSP181-SV1 G114S polymorph polypeptide”. The polypeptide having the sequence recited in SEQ ID NO:64 is hereafter referred to as “the mature his tag INSP181-SV1 G114S polymorph polypeptide”. The polypeptide having the sequence recited in SEQ ID NO:68 is referred to hereafter as “the lipocalin domain INSP181 his tag polypeptide” and comprises the lipocalin domain with a his tag. The polypeptide having the sequence recited in SEQ ID NO:72 is referred to hereafter as “the lipocalin domain INSP181-SV1 his tag polypeptide” and comprises the lipocalin domain of the splice variant with a his tag.

The term “INSP181 polypeptides” as used herein includes polypeptides comprising the INSP181 mature exon I polypeptide, the INSP181 polypeptide, the mature INSP181 polypeptide, the INSP181-SV1 polypeptide, the mature INSP-SV1 polypeptide, the his tag INSP181 polypeptide, the mature his tag INSP81 polypeptide, the his tag INSP181-SV1 polypeptide, the mature his tag INSP181-SV1 polypeptide, the INSP181 N92T polymorph polypeptide, the mature INSP181 N92T polymorph polypeptide, the INSP181-SV1-N92T polymorph polypeptide, the mature INSP181-SV1-N92T polymorph polypeptide, the his tag INSP181 N92T polymorph polypeptide, the mature his tag INSP181 N92T polymorph polypeptide, the his tag INSP181-SV1 N92T polymorph polypeptide, the mature his tag INSP181-SV1 N92T polymorph polypeptide, the his tag INSP181-SV1 G114S polymorph polypeptide, the INSP181-SV1 G114S polymorph polypeptide, the lipocalin domain INSP181 polypeptide and the lipocalin domain INSP181-SV1 polypeptide and his-tagged version. Polypeptides, including lipocalin domain polypeptides, including both the N92T and G114S polymorphisms are also included as aspects of the present invention.

INSP181 and INSP181-SV1 share the same predicted glycosylation site at amino acid 92 (FIG. 10), which is also the location of the predicted N92T polymorphism. Substitution of the asparagine at position 92 would prevent N-glycosylation. The presence or absence of sugar moieties may have important effects on the functioning of the INSP181 polypeptides.

We have also noted the presence of a disulphide bond formed between the cysteine amino acids at positions 90 and 181. Polypeptides of the invention in which these cysteine residues are disulphide-bonded are included as aspects of the present invention.

The term “lipocalin” refers to a molecule containing at least one lipocalin domain.

Preferably, the “lipocalin” may be a molecule containing a lipocalin domain detected with an e-value lower than 0.1, 0.01, 0.001, 0.0001, 0.0002, 0.00001, 0.000001 or 0.0000001.

Preferably, the term “lipocalin” may be a molecule matching the HMM build of the Pfam entry detected with an e-value lower than 0.1, 0.01, 0.001, 0.0001, 0.0002, 0.00001, 0.000001 or 0.0000001.

Preferably, a polypeptide according to any one of the above-described aspects of the invention functions as a lipocalin. Preferably, the lipocalin domain is encoded by residues 41-189 of the amino acid sequence of INSP181. The sequence of the lipocalin domain is given in SEQ ID NO: 66.

Further polypeptides according to the invention include fragments which maintain lipocalin activity and are polypeptides that comprise or consist of the lipocalin domain as shown in FIG. 14 (i.e. aa 25-174, or aa 26-180, or aa 33-166) and FIG. 11 (i.e. aa 41-189) as well as a fragment containing the cysteine residues forming the disulphide bond (i.e. aa 96-187).

A further polypeptide according to the invention is a lipocalin domain situated between residues 25 and 181 of INSP 181. For INSP181-SV1, the lipocalin domain is situated between residues 25 and 206, and comprises the sequence given in SEQ ID NO: 70.

By “functions as a lipocalin” we refer to polypeptides that comprise amino acid sequence or structural features that can be identified as conserved features within the polypeptides of the lipocalin family of proteins, such that the polypeptide's interaction with its biological partner is not substantially affected detrimentally in comparison to the function of the full length wild type polypeptide. In particular, we refer to the presence of cysteine residues in specific positions within the polypeptide that allow the formation of disulphide bonds.

Lipocalins are used as diagnostic and prognostic markers in a variety of disease states. The plasma level of AGP is monitored during pregnancy and in diagnosis and prognosis of conditions including cancer chemotherapy, renal disfunction, myocardial infarction, arthritis, and multiple sclerosis. Retinol-binding protein (RBP) is used clinically as a marker of tubular reabsorption in the kidney, and apo D is a marker in gross cystic breast disease.

Von Ebner's gland protein, is also known as tear lipocalin, tear prealbumin or VEGP. Similar to other lipocalins, VEGP is a carrier for retinol or other small hydrophobic compounds. VEGP binds retinol in vitro, and is believed to have an antimicrobial function in the eye, partly because it binds long chain fatty acids which inhibit activation of lysozyme (Glasgow, 1995 Arch. Clin. Exp. Opthalmol. 233:513-522). The protein may also inactivate enveloped viruses, help surface spreading of the lipid film in the eye and/or protect the epithelium.

Another member of the lipocalin family includes epididymal-retinoic acid binding protein (ERBP), which has tertiary structural homology to retinol-binding protein from human serum (Newcomer et al. 1990 J. Biol. Chem. 265:12876-12879). ERBP is believed to play and important role in maturation of the sperm as it passes through the epididymis. ERBP has been shown to bind a broad spectrum of retinoids, including retinol (vitamin A), retinal, retinyl acetate, beta-ionone, cis retinoids, beta-carotene, cholesterol, terpenoids, beta-lonylideneacetate, long-chain esters of retinol and retinoic acid (Flower, 1996 Biochem. J. 318:1-14) in vivo and/or in vitro. The retinoids have been demonstrated to play important roles in cell differentiation and proliferation, as well as vision, reproductive biology, and mucus secretion. For a review of retinoids and their role in disease and maintenance of homeostasis, see Goodman, D., 1984 N. Engl. J. Med. 310:1023-1031.

Prostaglandin D2 synthase is a lipocalin family member involved in the synthesis of prostaglandin D2 in the brain by catalyzing prostaglandin H2 into prostaglandin D2. Similar to other lipocalins, PD2 synthase is a carrier for hydrophobic compounds. PD2 synthase binds retinol in vitro, and has been proposed as a secretory retinoid transporter, that circulate retinoids in a variety of body fluids and transport them to their intracellular transporters. Once inside the cells, the retinoids bind to a dimerized receptor and ultimately play a biological role in the regulation of diverse processes, such as morphogenesis, differentiation, and mitogenesis (Tanaka et al., 1997, ibid.).

Other activities associated with members of the lipocalin family include antimicrobial, pheromone transport, modulators of inflammation, olfaction and regulation of immune response, regulation of nervous system development, and anti-bacterial activity.

One lipocalin associated with immune modulation is neutrophil gelatinase associated lipocalin (NGAL). NGAL has been localized to specific granules in neutrophils as both monomers and dimers (Bartsch et al., 1995 FEBS Letters 357: 255-259). NGAL is typical of lipocalins in that it binds small hydrophobic molecules to transport through hydrophilic fluids. While the physiological ligand for NGAL has not been identified, it has been shown to bind the bacterial chemotaxic factor FMLP, suggesting that the molecule binds lipophilic inflammatory mediators (Bungaard et al., 1994 Biochem. Biophys. Res. Comm. 202: 1468-1475).

The invention is based on the finding that polypeptides of the present invention upregulate Th2 cytokines, more specifically interleukin-10 (IL-10), interleukin-4 (IL-4) and interleukin-5 (IL-5). In addition, it has been surprisingly found that the polypeptides of the present invention show unexpected restricted expression in skin biopsy samples and particularly in psoriasis skin biopsies.

Immune diseases can be divided into those characterized by T helper cell 1 (Th1) or T helper cell 2 (Th2) dominance. Drugs may affect the Th1/Th2 balance with the aim of modifying the autoimmune disease in question.

A Th1 disease is herein defined as a disease selected from Crohn's disease, Diabetes type 1, Hashimoto's disease, Grave's disease (thyroiditis), psoriasis, a Th1 skin disease such as psoriasis or hyperkeratotic dermatose, rheumatoid arthritis, proliferative and crescentic forms of glomerulonephritis, multiple sclerosis, posterior uveitis, wound healing, and/or sarcoidosis.

Preferably, the Th1 skin disease is psoriasis.

Preferably, the hyperkeratotic dermatose is selected from psoriasis, pityriasis rubra and/or porokeratosis.

A Th2 disease is herein defined as a disease selected from allergies such as allergic rhinitis, asthma, a Th2 skin disease, lichen rubber planus, chronic sinusitis, Sezary syndrome, cancer, actinic keratosis, hepatitis C, ulcerative colitis, membranous glomerulonephritis and/or viral infections.

Preferably, the Th2 skin disease is selected from atopic dermatitis, contact dermatitis, contact allergy to e.g. nickel or gold, cutaneous T-cell lymphoma, atopic eczema, acute eczema and/or chronic eczema.

Preferably, the atopic dermatitis is an acute atopic dermatitis.

Preferably, the cancer is selected from cutaneous T-cell lymphoma, squamous cell carcinoma and/or basal cell carcinoma.

Without wishing to be bound to this theory, a polypeptide of the present invention, alone or as part of a fusion protein, and/or a fragment thereof may switch the T cell cytokine bias from Th1 to Th2.

As such, a polypeptide of the present invention, alone or as part of a fusion protein, and/or a fragment thereof is useful for the treatment of a Th1 disease.

Without wishing to be bound to this theory, an antagonist, for example an antibody directed to a polypeptide of the invention, may switch the T cell cytokine bias from Th2 to Th1.

As such an antagonist, for example an antibody directed to a polypeptide of the invention, is useful for the treatment of a Th2 disease. For example, an enhanced 2 immune response and the elaboration of cytokines such as IL-4, IL-5 and IL-13 contribute to the induction of allergy and asthma (Ngoc et al. Curr Opin Allergy Clin Immunol. 2005 April; 5(2):161-6.). As such, an antagonist, for example an antibody directed to a polypeptide of the invention, is useful for the treatment of asthma and/or allergy.

Apart from targeting cellular mechanisms, approaches in psoriasis therapy work via humoral immunomodulation by influence of the type I dominated cytokine imbalance with elevated levels of IL-2, -6, -8, TNF-α or IFN-γ. This can be achieved by administration of exogenous deficient counter-regulating type 2 cytokines such as IL-4, -10 and -11 to deviate the differentiation of type 1 cytokine-producing T cells potentially to the production of type 2 cytokines, which stimulate endogenous differentiation of type 2 T lymphocytes in normal immune response. Without wishing to be bound to this theory, the polypeptides of the present invention are thought likely to work in a similar way.

Drugs modulating cytokine production such as IL-4 or IL-10 are known and administration of recombinant cytokines such as IL-4, IL-10 and IL-11 are being considered for the treatment of psoriasis (Schleyer et al. J Eur Acad Dermatol Venereol. 2005 January; 19(1):1-20).

For example, an initial phase 1b study with different doses of recombinant IL-4 showed impressive antipsoriatic effects (Schleyer et al.). Twenty of 22 patients accrued to five different doses of weekly IL-4 injections over a 6-week treatment period terminated the study and one patient showed grade II adverse events. In 18 patients PASI decreased by 60-80% within 6 weeks and no rebound occurred during a 6-week follow-up. Improvement in psoriasis was dose dependent, associated with a decrease in skin infiltrating cells and normalization of the epidermal structure. These early data suggest that this could be a very successful approach to the treatment of psoriasis by immune deviation, affecting exclusively the recently activated T cells.

In psoriatic lesions there is a relative deficiency in cutaneous IL-10 expression. This Th2 cytokine is a potent inhibitor of APC functions such as T-cell proliferation. It also suppresses type 1 cytokine production including IFN-γ or TNF-α and therefore plays an essential role in the control of inflammatory skin responses.

Like conventional therapies such as fumaric ester derivates, topical vitamin D3 analogues and UV-irradiation which work, among others, via an upregulation of endogenous IL-10, systemic administration of IL-10 was thought to be effective in the treatment of psoriasis by relative restoration of the cytokine balance.

Studies have indicated that the lipocalins may be useful for the treatment of the following diseases: vision disorders (e.g. nightblindness), immune system disorders (e.g. autoimmune disorders), inflammatory disorders, inflammatory bowel disease (IBD), ulcerative colitis (UC), Crohn's disease (CD), proctitis, cell proliferative disorders, cancer (e.g. breast cancer, cutaneous T-cell lymphoma, squamous cell carcinoma and/or basal cell carcinoma), microbial infections (e.g. viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to e.g. nickel or gold, cutaneous T-cell lymphoma, atopic eczema, acute eczema and/or chronic eczema), reproductive disorders (e.g. infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis and/or viral infections.

It is postulated that INSP181 might belong to the immunocalin subfamily and share the functionalities of the immunocalin members, more particularly with glycodelin (see, for a review, Lögdberg and Wester. Biochim Biophys Acta. 2000 1482(1-2):284-97). Family members are encoded by a cluster of genes in the q32-34 region of chromosome 9 in the human genome (INSP181) in the q34 region). Glycodelin has been implicated in fertilization, immunomodulation and differentiation. Three major isoforms of glycodelin can be detected (GdA, GdS and GdF), conferring specific functionalities and highlighting the importance of glycosylation for biological activity in the immunocalin subfamily.

WO02/053701 discloses lipocalin nucleic acids and polypeptides (more particularly human EP17 gene) that can be used to generate a mouse model of male infertility, for drug discovery screens, and for therapeutic treatment of fertility-related conditions. DE19807389 discloses monoclonal antibodies against glycodelin A useful for the treatment of cancer.

Preferably, the activity of a polypeptide of the present invention, alone or as part of a fusion protein, a fragment thereof and/or an antagonist thereof can be confirmed in at least one of the following assays:

a) in models of skin cancers as reviewed by Odashiro et al. (Drug Discovery Today: Disease Models 2005; in press), or
b) in models of contact dermatitis or atopic eczema as reviewed by Gutermuth et al. (Drug Discovery Today: Disease Models 2005, in press), or
c) in models of atopic dermatitis as reviewed by Zheng and Zhu (Curr Allergy Asthma Rep. 2005 July; 5(4):291-7), or
d) in the D6 mouse model as disclosed by Jamieson et al. (Nat Immunol. 2005 April; 6(4):403-11), or
e) in the assay measuring cytokine secretion by Con A stimulated PBMCs as described in Example 5. More specifically, the activity of a polypeptide of the present invention, alone or as part of a fusion protein, a fragment thereof and/or an antagonist thereof can be confirmed if at least one of the following cytokines are modulated: IL-4, IL-5 and/or IL-10. Preferably, two (e.g. IL-4 and IL-5; IL-4 and IL-10; or IL-5 and IL-10) or all three cytokines are modulated. Preferably, a polypeptide of the present invention, alone or as part of a fusion protein, and/or a fragment thereof upregulate one or more of the above-mentioned cytokines. Preferably, an antagonist, for example an antibody directed to a polypeptide of the invention, downregulate one or more of the above-mentioned cytokines.

Activity of the polypeptides of the invention, e.g. INSP181, can be evaluated by any of the methods described in the invention or known in the art.

The polypeptides of the first aspect of the invention may further comprise a histidine tag. Preferably the histidine tag is found at the C-terminal of the polypeptide. Preferably the histidine tag comprises 1-10 histidine residues (e.g. 1, 2, 3, 4, 5, 6, 7, 8, 9 or 10 residues). More preferably the histidine tag comprises 6 histidine residues.

An “antigenic determinant” of the present invention may be a part of a polypeptide of the present invention, which binds to an antibody-combining site or to a T-cell receptor (TCR). Alternatively, an “antigenic determinant” may be a site on the surface of a polypeptide of the present invention to which a single antibody molecule binds. Generally an antigen has several or many different antigenic determinants and reacts with antibodies of many different specificities. Preferably, the antibody is immunospecific to a polypeptide of the invention. Preferably, the antibody is immunospecific to a polypeptide of the invention, which is not part of a fusion protein. Preferably, the antibody is immunospecific to INSP181, INSP181-SV or a fragment thereof. Antigenic determinants usually consist of chemically active surface groupings of molecules, such as amino acids or sugar side chains, and can have specific three dimensional structural characteristics, as well as specific charge characteristics. Preferably, the “antigenic determinant” refers to a particular chemical group on a polypeptide of the present invention that is antigenic, i.e. that elicit a specific immune response.

In a second aspect, the invention provides a purified nucleic acid molecule which encodes a polypeptide of the first aspect of the invention.

The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “purified nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. In a preferred embodiment, genomic DNA are specifically excluded from the scope of the invention. Preferably, genomic DNA larger than 10 kbp (kilo base pairs), 50 kbp, 100 kbp, 150 kbp, 200 kbp, 250 kbp or 300 kbp are specifically excluded from the scope of the invention. Preferably, the “purified nucleic acid molecule” consists of cDNA only.

Preferably, the purified nucleic acid molecule consists or comprises the nucleic acid sequence as recited in SEQ ID NO:1 (encoding the INSP181 exon 1 polypeptide), SEQ ID NO:3 (encoding the INSP181 exon 2 polypeptide), SEQ ID NO:5 (encoding the INSP181 exon 3 polypeptide), SEQ ID NO:7 (encoding the INSP181-SV1 exon 3 polypeptide), SEQ ID NO:9 (encoding the INSP181-SV1 exon 4 polypeptide), SEQ ID NO:11 (encoding the INSP181 exon 4 polypeptide), SEQ ID NO:13 (encoding the INSP181 exon 5 polypeptide), SEQ ID NO:15 (encoding the INSP181 exon 6 polypeptide), SEQ ID NO:17 (encoding the INSP181 polypeptide), SEQ ID NO:19 (encoding the INSP181 mature exon 1 polypeptide), SEQ ID NO:21 (encoding the INSP181 mature polypeptide), SEQ ID NO:23 (encoding the INSP181-SV1 polypeptide), SEQ ID NO:22 (encoding the mature INSP181-SV1 polypeptide), SEQ ID NO:27 (encoding the his tag INSP181 polypeptide), SEQ ID NO:29 (encoding the mature his tag INSP181 polypeptide), SEQ ID NO:31 (encoding the his tag INSP181-SV1 polypeptide), SEQ ID NO:33 (encoding the mature his tag INSP181-SV1 polypeptide), SEQ ID NO:35 (encoding INSP181-exon 3 N92T polymorph polypeptide), SEQ ID NO:37 (encoding the INSP181-SV1 exon 3 N92T polymorph polypeptide), SEQ ID NO:39 (encoding the INSP181-N92T polymorph polypeptide), SEQ ID NO:41 (encoding the mature INSP181-N92T polymorph polypeptide), SEQ ID NO:43 (encoding the INSP181-SV1 N92T polymorph polypeptide), SEQ ID NO:45 (encoding the mature INSP181-SV1 N92T polymorph polypeptide), SEQ ID NO:47 (encoding the his tag INSP181 N92T polymorph polypeptide), SEQ ID NO:49 (encoding the mature his tag INSP181 N92T polymorph polypeptide), SEQ ID NO:51 (encoding the his tag INSP181-SV1 N92T polymorph polypeptide), SEQ ID NO:53 (encoding the mature his tag INSP181-SV1 N92T polymorph polypeptide), SEQ ID NO:55 (encoding the INSP181-SV1 exon 4 G114S polymorph polypeptide), SEQ ID NO:57 (encoding the INSP181-SV1 G114S polymorph polypeptide), SEQ ID NO:59 (encoding the mature INSP181-SV1 G114S polymorph polypeptide), SEQ ID NO:61 (encoding the his tag INSP181-SV1 G114S polymorph polypeptide), SEQ ID NO:63 (encoding the mature his tag INSP181-SV1 G114S polymorph polypeptide), SEQ ID NO:65 (the alternative exon 2 nucleotide), SEQ ID NO:67 (encoding the lipocalin domain INSP181 polypeptide), SEQ ID NO:69 (encoding the lipocalin domain INSP181 his tag polypeptide), SEQ ID NO: 71 (encoding the lipocalin domain INSP181-SV1 polypeptide) and/or SEQ ID NO: 73 (encoding the lipocalin domain INSP181-SV1 his tag polypeptide)

In a third aspect, the invention provides a purified nucleic acid molecule which hydridizes under high stringency conditions with a nucleic acid molecule of the second aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the ability of a polypeptide of the first aspect of the invention to transport small, hydrophobic molecules.

Ligands to a polypeptide according to the invention may come in various forms, including natural or modified substrates, enzymes, receptors, small organic molecules such as small natural or synthetic organic molecules of up to 2000Da, preferably 800Da or less, peptidomimetics, inorganic molecules, peptides, polypeptides, antibodies, structural or functional mimetics of the aforementioned.

Such compounds may be identified using the assays and screening methods disclosed herein.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide.

Importantly, the identification of the function of the INSP181 polypeptides allows for the design of screening methods capable of identifying compounds that are effective in the treatment and/or diagnosis of disease. Ligands and compounds according to the sixth and seventh aspects of the invention may be identified using such methods. These methods are included as aspects of the present invention.

Compounds identified as agonists of the polypeptides of the invention may be useful for transportation of small hydrophobic molecules either in vitro or in vivo. For example, agonist compounds are useful as components of defined cell culture media, to deliver small, hydrophobic molecules to cells and protect them from degradation by enzymes present in serum.

Antagonists (e.g. antibodies) of INSP181, INSP181-SV1, INSP181 N92T polymorph, INSP181-SV1 N92T polymorph and/or INSP181-G114S polymorph might be useful in the treatment of cancer, more particularly cancer affecting the brain, the ovaries, the testis, the spleen, the pancreas, the uterus, the blood and/or the lung.

Preferably, antagonists (e.g. antibodies) of INSP181-SV1, INSP181-SV1 N92T polymorph or INSP181-SV1 G114S polymorph (derived from salivery gland, adrenal gland and eye cDNA) might be useful in the treatment of cancer, more particularly cancers affecting the salivery glans, adrenal gland and/or eye.

Another aspect of this invention resides in the use of an INSP181 gene or polypeptide as a target for the screening of candidate drug modulators, particularly candidate drugs active against lipocalin related disorders.

A further aspect of this invention resides in methods of screening of compounds for therapy of lipocalin related disorders, comprising determining the ability of a compound to bind to an INSP181 gene or polypeptide, or a fragment thereof.

A further aspect of this invention resides in methods of screening of compounds for therapy of lipocalin related disorders, comprising testing for modulation of the activity of an INSP181 gene or polypeptide, or a fragment thereof.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis.

The moeities of the invention may be used in the manufacture of a medicament for the treatment of certain diseases including, but not limited to vision disorders (e.g. nightblindness), immune system disorders (e.g. autoimmune disorders), inflammatory disorders, inflammatory bowel disease (IBD), ulcerative colitis (UC), Crohn's disease (CD), proctitis, cell proliferative disorders, cancer (e.g. breast cancer, cutaneous T-cell lymphoma, squamous cell carcinoma and/or basal cell carcinoma), microbial infections (e.g. viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to e.g. nickel or gold, cutaneous T-cell lymphoma, atopic eczema, acute eczema and/or chronic eczema), reproductive disorders (e.g. infertility, in particular male infertility), renal dysfunction, myocardial infarction, arthritis, gross cystic breast disease, regulation of nervous system development, Diabetes type 1, Hashimoto's disease, Grave's disease (thyroiditis), rheumatoid arthritis, proliferative and crescentic forms of glomerulonephritis, multiple sclerosis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis and/or viral infections.

The assays set forth in the Examples may also be useful for the identification of therapeutically useful moieties.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over a period of time towards a control level is indicative of regression of disease.

Higher expression of lipocalins can be detected in patients suffering from skin diseases such as psoriasis compared to unaffected patients or when patients are exposed to allergens. For example, in patients exposed to nickel or gold (contact allergy), a significant increase in neutrophil gelatinase associated lipocalin (NGAL) has been measured (Moller et al. Contact Dermatitis. 1999 April; 40(4):200-4;). Upregulation of peptides/polypeptides such as NGAL was also detected after wound healing and offer an explanation for the expression of these peptides/polypeptides in psoriasis and wound healing (Sorensen et al. J Immunol. 2003 Jun. 1; 170(11):5583-9.). In addition, strong induction of NGAL in the epidermis was seen in a variety of skin disorders characterized by dysregulated epithelial differentiation such as psoriasis, pityriasis rubra and squamous cell carcinoma (Mallbris et al. Exp Dermatol. 2002 December; 11(6):584-91). Thus, Mallbris et al. conclude that NGAL is a marker for dysregulated keratinocyte differentiation in human skin.

Thus, overexpression of certain polypeptides of the present invention may correlate with disease progression and prognosis. As such, polypeptides of the present invention are useful as markers for disease progression and/or prognosis.

It is believed that certain tissues in mammals with Th1 disease or Th2 disease contain significantly greater INSP181 protein gene copy number and express significantly enhanced levels of the INSP181 protein and mRNA encoding the INSP181 protein when compared to a corresponding “standard” mammal, i.e., a mammal of the same species not having the Th1 disease or Th2 disease. Enhanced levels of the INSP181 protein will be detected in certain body fluids (e.g., sera, plasma, urine, synovial and spinal fluid) and/or in the skin from mammals with Th1 disease or Th2 disease when compared to sera/skin from mammals of the same species not having the Th1 disease or Th2 disease. Thus, the invention provides a method useful during Th1 disease or Th2 disease diagnosis, which involves assaying the expression level of the gene encoding the INSP181 protein or the gene copy number in mammalian cells, particularly in skin, or body fluid and comparing the gene expression level or gene copy number with a standard INSP181 protein gene expression level or gene copy number, whereby an increase in the gene expression level or gene copy number over the standard is indicative of certain Th1 diseases or Th2 diseases.

Where a Th1 disease or Th2 disease diagnosis has already been made according to conventional methods, the present invention is useful as a prognostic indicator.

By “assaying the expression level of the gene encoding the INSP181 protein” is intended qualitatively or quantitatively measuring or estimating the level of the INSP181 protein or the level of the mRNA encoding the INSP181 in a first biological sample either directly (e.g., by determining or estimating absolute protein level or mRNA level) or relatively (e.g., by comparing to the INSP181 protein level or mRNA level in a second biological sample). By “assaying the copy number of the gene encoding the INSP181 protein” is intended qualitatively or quantitatively measuring or estimating the gene copy number in a first biological sample either directly (e.g., by determining or estimating absolute gene copy number) or relatively (e.g., by comparing to the INSP181 protein gene copy number in a second biological sample).

Preferably, the INSP181 protein level, mRNA level, or gene copy number in the first biological sample is measured or estimated and compared to a standard INSP181 protein level, mRNA level, or gene copy number, the standard being taken from a second biological sample obtained from an individual not having the Th1 disease or Th2 disease. Alternatively, where the method is used as a prognostic indicator, both the first and second biological samples can be taken from individuals having the Th1 disease or Th2 disease and the relative expression levels or copy number will be measured to determine prognosis. As will be appreciated in the art, once a standard INSP181 protein level, mRNA level, or gene copy number is known, it can be used repeatedly as a standard for comparison.

By “biological sample” is intended any biological sample obtained from an individual, cell line, tissue culture, or other source which contains INSP181 protein or mRNA, preferably from the skin. Methods for obtaining tissue biopsies and body fluids from mammals are well known in the art. Where the biological sample is to include mRNA, a tissue biopsy is the preferred source.

The present invention is useful for detecting Th1 disease or Th2 disease in mammals. In particular the invention is useful during diagnosis or prognosis in mammals of the types of Th1 disease or Th2 disease mentioned in the present invention. Preferred mammals include monkeys, apes, cats, dogs, cows, pigs, horses, rabbits and humans. Particularly preferred are humans.

One possible method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a lipocalin.

The polypeptides of the present invention might be used for binding small fatty acids, for instance in blood or tissues to modulate their biological function. The polypeptides of the present invention could be used to transport retinoids or steroids to receptors, in particular as part of the therapy for breast cancer, emphysema and diseases of the skin and play and important role in reproduction. Other uses include modulation of anti-inflammatory responses, activity as a microbial, either as an enhancer of enzyme function or as an enzyme-like molecule itself.

The polypeptides of the present invention might be useful for their antimicrobial properties. Antimicrobial activity can be measured in vitro using cultured cells or in vivo by administering molecules of the claimed invention to the appropriate animal model. Assays for testing antimicrobial activity are specific to the microbe and are generally known by those ordinarily skilled in the art. For example, in vivo testing for antimicrobial activity is done by inoculating mice intraperitoneally with pathogenic microorganisms in an appropriate broth. Shortly after inoculation, a composition containing the polypeptide is administered and death during the subsequent 7 days is recorded. Generally administration is intravenous, subcutaneous, intraperitoneal or by mouth. See, for example, Musiek et al., Antimicrobial Agents Chemother. 3:40, 1973, for discussion of in vivo and in vitro testing of antimicrobials.

The activity of polypeptides of the present invention can be measured using a variety of assays that measure the ability to bind small hydrophobic molecules. Such assays include, but are not limited to assays measuring changes in fluorescence intensity (Cogan et al., Eur. J. Biochem. 65:71-78, 1976) and equilibrium dialysis of water soluble compounds (Hase et al., J. Biochem. 79:373-380, 1976).

Other utilities for molecules of the present invention include as a delivery system to transport and/or stabilize small lipophilic molecules. For example, molecules of the present invention could be used to microencapsulate a small lipophilic molecule that forms an active pharmacological agent, and thus protect the agent from extreme pH in the gut, exposure to powerful digestive enzymes and impermeability of gastrointestinal membranes to the active ingredient. Other advantages as encapsulation of the pharmacologic agent can include preventing premature activation of the agent or protection from gastric irritants.

Recently, the lipocalin scaffold was used to engineer proteins with tailored specificity for non-natural ligands. Such designed lipocalins can be considered as antibody mimics and have thus been named “anticalins” (for a review, see Skerra, Biochim Biophys Acta. 2000 Oct. 18; 1482(1-2):337-50.). Accordingly, the polypeptides of the invention might find utility in the synthesis of “anticalins”.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease including vision disorders (e.g. nightblindness), immune system disorders (e.g. autoimmune disorders), inflammatory disorders, inflammatory bowel disease (IBD), ulcerative colitis (UC), Crohn's disease (CD), proctitis, cell proliferative disorders, cancer (e.g. breast cancer), microbial infections (e.g. viral, bacterial and fungal infections), emphysema, skin diseases, reproductive disorders (e.g. infertility, in particular male infertility), renal dysfunction, myocardial infarction, arthritis, and multiple sclerosis, gross cystic breast disease and regulation of nervous system development.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

Polypeptides of the invention are useful on their own, as components of fusion proteins such as Fc fusion, and/or in combination with one or more agent acting as down regulator of Th1. Antagonists, for example an antibody directed to a polypeptide of the invention, can be used on their own or in combination with one or more agent acting as down regulator of Th2.

Agents acting as down regulators of Th1 or Th2 are well known in the Art and should not be limited to the below-mentioned agents.

Preferably, the agent acting as down regulator of Th1 is selected from cellular immunomodulators, humoral immunomodulators, polypeptide T, mycobacterium vaccae, tazarotene, bexarotene, troglitazone, liarozole, rambazole, arginine, nitric oxide, cyclosporin, methotrexate, Vitamin D3 analogues, retinoids, corticosteroids, anthralin, tar, Psoralen plus UVA (PUVA), curcumin, polypodium, leucotomas, glucocorticoid such as predisone, and/or balancer of Th1/Th2 (adaptogens) such as soy isoflavones, plant sterols, sterolins, probiotics and/or pregnenalone.

Preferably, the cellular immunomodulator is selected from DAB389IL-2, mycophenolate mofetil, VX-497, leflunomide, efalizumab, OKTcdr4a, CTLA4-Ig, MEDI 507, LFA3TIP, daclizumab, basiliximab, tacrolimus, pimecrolimus and/or sirolimus.

Preferably, the humoral immunomodulator is selected from IL-4, IL-10, IL-11, infliximab, etanercept, onercept and/or adalimumab.

Preferably the agent acting as down regulator of Th2 is selected from antagonists (e.g. antibodies) to the chemokine receptor CCR3 or CCR4, CXCR4 antagonists, anti-TARC, inhibitors of the adhesion molecule VLA-4, PPAR-γ agonists, cyclopentenone prostaglandins, thiazolidinediones, SB203580, SB239063, RWJ67657, Vitamin D3 analogues, glucocorticoids, mycobacteria, anti-IL-5/IL-13/IL-9, soluble IL-4R, inhibitors of CD80/86, ICOS ligand, a Toll-like receptor (TLR)-9 agonist such as CpG DNA, CTLA4-Ig, antisense GATA3 oligonucleotides, mycobacterium bacillus Calmette-Guerin (BCG), mycobacterium vaccae, anti-IgE, beta receptor agonists, corticosteroids, perilla seed, quercetin, luteolin, isoflavones, glucocorticoid such as predisone, and/or balancer of Th1/Th2 (adaptogens) such as soy isoflavones, plant sterols, sterolins, probiotics and/or pregnenalone. For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

The INSP181 polypeptides are lipocalins and thus have roles in many disease states. Antagonists of the INSP181 polypeptides are of particular interest as they provide a way of modulating these disease states.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be using in screening regimes for the identification of compounds that are effective in the treatment or diagnosis of such a disease.

As used herein, “functional equivalent” refers to a protein or nucleic acid molecule that possesses functional or structural characteristics that are substantially similar to a polypeptide or nucleic acid molecule of the present invention. A functional equivalent of a protein may contain modifications depending on the necessity of such modifications for the performance of a specific function. The term “functional equivalent” is intended to include the fragments, mutants, hybrids, variants, analogs, or chemical derivatives of a molecule.

Preferably, the “functional equivalent” may be a protein or nucleic acid molecule that exhibits any one or more of the functional activities of the polypeptides of the present invention.

Preferably, the “functional equivalent” may be a protein or nucleic acid molecule that displays substantially similar activity compared with INSP181 or fragments thereof in a suitable assay for the measurement of biological activity or function. Preferably, the “functional equivalent” may be a protein or nucleic acid molecule that displays identical or higher activity compared with INSP181 or fragments thereof in a suitable assay for the measurement of biological activity or function. Preferably, the “functional equivalent” may be a protein or nucleic acid molecule that displays 50%, 60%, 70%, 80%, 90%, 95%, 98%, 99%, 100% or more activity compared with INSP181- or fragments thereof in a suitable assay for the measurement of biological activity or function.

Preferably, the “functional equivalent” may be a protein or polypeptide capable of exhibiting a substantially similar in vivo or in vitro activity as the polypeptides of the invention. Preferably, the “functional equivalent” may be a protein or polypeptide capable of interacting with other cellular or extracellular molecules in a manner substantially similar to the way in which the corresponding portion of the polypeptides of the invention would. For example, a “functional equivalent” would be able, in an immunoassay, to diminish the binding of an antibody to the corresponding peptide (i.e., the peptide the amino acid sequence of which was modified to achieve the “functional equivalent”) of the polypeptide of the invention, or to the polypeptide of the invention itself, where the antibody was raised against the corresponding peptide of the polypeptide of the invention. An equimolar concentration of the functional equivalent will diminish the aforesaid binding of the corresponding peptide by at least about 5%, preferably between about 5% and 10%, more preferably between about 10% and 25%, even more preferably between about 25% and 50%, and most preferably between about 40% and 50%.

For example, functional equivalents can be fully functional or can lack function in one or more activities. Thus, in the present invention, variations can affect the function, for example, of the activities of the polypeptide that reflect its possession of a lipocalin domain.

A summary of standard techniques and procedures which may be employed in order to utilise the invention are given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

In particular, the fusion protein may comprise one or more additional amino acid sequences and a fragment of the polypeptides of the present invention. The fragment is preferably a lipocalin domain, for example as recited in SEQ ID NO: 66, SEQ ID NO: 70, or amino acids 25-174, amino acids 26-180, amino acids 33-166, or amino acids 41-189 of SEQ ID NO: 18 or amino acids 25-206 of SEQ ID NO:24.

Preferably, the polypeptide of the invention comprising a sequence having at least 85% homology with an INSP181 polypeptide is a fusion protein. Such fusion proteins can be obtained by cloning a polynucleotide encoding a polypeptide comprising a sequence having at least 85% homology with an INSP181 polypeptide in frame with the coding sequences for a heterologous protein sequence.

The term “heterologous”, when used herein, is intended to designate any polypeptide other than a human INSP181 polypeptide. Examples of heterologous sequences, that can be comprised in the fusion proteins either at the N- or C-terminus, include: extracellular domains of membrane-bound protein, immunoglobulin constant regions (Fc regions), multimerization domains, domains of extracellular proteins, signal sequences, export sequences, and sequences allowing purification by affinity chromatography.

Many of these heterologous sequences are commercially available in expression plasmids since these sequences are commonly included in fusion proteins in order to provide additional properties without significantly impairing the specific biological activity of the protein fused to them (Terpe K, 2003, Appl Microbiol Biotechnol, 60:523-33). Examples of such additional properties are a longer lasting half-life in body fluids, the extracellular localization, or an easier purification procedure as allowed by the a stretch of Histidines forming the so-called “histidine tag” (Gentz et al. 1989, Proc Natl Acad Sci USA, 86:821-4) or by the “HA” tag, an epitope derived from the influenza hemagglutinin protein (Wilson et al. 1994, Cell, 37:767-78). If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the protein used in the examples (the mature INSP181 polypeptide; SEQ ID NO: 22) was purified by means of a hexa-histidine peptide fused at the C-terminus of INSP181. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (i.e. an increased half-life), increased specific activity, increased expression level, or the purification of the fusion protein is facilitated.

In a preferred embodiment, the protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP181 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP181 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues includes a substituent group.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table 1.

Specific, non-conservative mutations can be also introduced in the polypeptides of the invention with different purposes. Mutations reducing the affinity of the CD24-like protein may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table 2. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem A G, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Typically, greater than 30% identity between two polypeptides is considered to be an indication of functional equivalence. Preferably, functionally equivalent polypeptides of the first aspect of the invention have a degree of sequence identity with the INSP181 polypeptides, or with active fragments thereof, of greater than 70% or 80%. More preferred polypeptides have degrees of identity of greater than 85%, 90%, 95%, 98%, 98.5%, 99% or 99.5% respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium search database may be used (see WO 01/67507) to identify polypeptides of presently-unknown function which, while having low sequence identity as compared to the INSP181 exon polypeptides or the INSP181 polypeptide (SEQ ID NOs. 14 and 18), are predicted to be lipocalins, by virtue of sharing significant structural homology with the INSP181 exon polypeptides, the INSP181 polypeptide, the INSP181-SV1 polypeptide, the INSP181-SV1 polypeptide, the INSP181 mature polypeptide, the INSP181-SV1 mature polypeptide, the INSP181 N92T polymorph polypeptide, the mature INSP181 polymorph polypeptide, the INSP181-SV1 N92T polymorph polypeptide, the mature INSP181 N92T polymorph polypeptide, the INSP181-SV1G114S polymorph polypeptide or the mature INSP181-SV1 G114S polymorph polypeptide.

By “significant structural homology” is meant that the Inpharmatica Genome Threader™ predicts two proteins, or protein regions, to share structural homology with a certainty of at least 10% more preferably, at least 20%, 30%, 40%, 50%, 60%, 70%, 80%, 90% and above. The certainty value of the Inpharmatica Genome Threader™ is calculated as follows. A set of comparisons was initially performed using the Inpharmatica Genome Threader™ exclusively using sequences of known structure. Some of the comparisons were between proteins that were known to be related (on the basis of structure). A neural network was then trained on the basis that it needed to best distinguish between the known relationships and known not-relationships taken from the CATH structure classification (www.biochem.ucl.ac.uk/bsm/cath). This resulted in a neural network score between 0 and 1. However, again as the number of proteins that are related and the number that are unrelated were known, it was possible to partition the neural network results into packets and calculate empirically the percentage of the results that were correct. In this manner, any genuine prediction in the Biopendium search database has an attached neural network score and the percentage confidence is a reflection of how successful the Inpharmatica Genome Threader™ was in the training/testing set.

The polypeptides of the first aspect of the invention also include fragments of the INSP181 polypeptides and fragments of the functional equivalents of the INSP181 polypeptides, provided that those fragments are lipocalins or have an antigenic determinant in common with the INSP181 polypeptide, INSP181 mature polypeptide, the INSP181-SV1 polypeptide, the INSP181-SV1 mature polypeptide, the INSP181 N92T polymorph polypeptide, the mature INSP181 polymorph polypeptide, the INSP181-SV1 N92T polymorph polypeptide, the mature INSP181 N92T polymorph polypeptide, the INSP181-SV1G114S polymorph polypeptide, the mature INSP181-SV1 G114S polymorph polypeptide, the INPS181 lipoclain domain or the INSP181-SV1 lipoclain domain.

Examples of fragments that maintain lipocalin activity are those comprising or consisting of the lipocalin domain, as given in SEQ ID NO: 66, or sequences as shown in FIG. 12 (i.e. amino acids 25-174, amino acids 26-180 and/or amino acids 33-166 of any of the full length INSP181 sequences), as well as a fragment containing the cysteine residues forming the disulphide bond (i.e. amino acids 96-187 of any of the full length INSP181 sequences). Preferably, the disulphide bond is formed between the cysteine amino acids at positions 90 and 181.

As used herein, the term “fragment” refers to a polypeptide having an amino acid sequence that is the same as part, but not all, of the amino acid sequence of the INSP181 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant. Fragments according to the invention may be 1-100 amino acids in length, preferably, 5-50, more preferably 7-20 amino acids.

Nucleic acids according to the invention are preferably 10-1000 nucleotides in length, preferably 50-800 nucleotides, preferably 100-600, preferably 200-550, preferably 300-500 nucleotides in length. Polypeptides according to the invention are preferably 5-500 amino acids in length, preferably 50-400, preferably 100-300, preferably 150-250 amino acids in length.

Fragments of the full length INSP181 polypeptides may consist of combinations of 1 or 2, 3, 4, 5 . . . neighbouring exon sequences in the INSP1813 polypeptide sequences, respectively. These exons may be combined with further mature fragments according to the invention. For example, such combinations include exons 1 and 2, and so on. Such fragments are included in the present invention. Fragments may also consist of combinations of different domains of the INSP181 protein. For example a fragment may consist of combinations of the different lipocalin domains of INSP181 as recited above. Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for other related polypeptides in the prior art such as known lipocalins.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for other related polypeptides in the prior art.

Preferably, there is a measurable increase in the affinity for a polypeptide of the invention as compared with known lipocalins.

Preferably, there is a measurable increase in the affinity for a polypeptide of the invention as compared with natural lipocalin.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet hemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is, an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode the polypeptide sequences recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO: 66 or SEQ ID NO:68 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO: 66 or SEQ ID NO: 68 and functionally equivalent polypeptides.

Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally occurring variant such as a naturally occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding an INSP181 polypeptide (SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO: 66 and SEQ ID NO: 68), and nucleic acid molecules that are substantially complementary to these nucleic acid molecules. Preferably, a nucleic acid molecule according to this aspect of the invention comprises a region that is at least 80% identical over its entire length to a nucleic acid molecule having the sequence given in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, or SEQ ID NO: 69 or a nucleic acid molecule that is complementary thereto. In this regard, nucleic acid molecules at least 90%, preferably at least 95%, more preferably at least 98%, 98.5%, 99% or 99% identical over their entire length to the same are particularly preferred. Preferred embodiments in this respect are nucleic acid molecules that encode polypeptides which retain substantially the same biological function or activity as the INSP181 polypeptide, the INSP181 mature polypeptide, the INSP-SV1 polypeptide, the INSP181-SV1 mature polypeptide, the INSP181 N92T polymorph polypeptide, the mature INSP181 polymorph polypeptide, the INSP181-SV1 N92T polymorph polypeptide, the mature INSP181 N92T polymorph polypeptide, the INSP181-SV1G114S polymorph polypeptide, the mature INSP181-SV1 G114S polymorph polypeptide or the lipocalin domain INSP181 polypeptide.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP181 polypeptides and to isolate cDNA and genomic clones of homologous or orthologous genes that have a high sequence similarity to the gene encoding these polypeptides.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP181 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67 and/or SEQ ID NO:69) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfested or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The vectors pCR4-TOPO-INSP181, pCR4-TOPO-INSP181-SV1, pDONR221_INSP181-6HIS, pDONR221_INSP181SV1-6HIS, pEAK_INSP181-6HIS, pEAK_INSP181SV1-6HIS, pDEST12.2_INSP181-6HIS, and pDEST12.2_INSP181SV1-6HIS are preferred examples of suitable vectors for use in accordance with the aspects of this invention relating to INSP181.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., [supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture have been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk− or aprt± cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinothricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

As indicated above, the present invention also provides novel targets and methods for the screening of drug candidates or leads. These screening methods include binding assays and/or functional assays, and may be performed in vitro, in cell systems or in animals.

In this regard, a particular object of this invention resides in the use of an INSP181 polypeptide as a target for screening candidate drugs for treating or preventing lipocalin related disorders.

Another object of this invention resides in methods of selecting biologically active compounds, said methods comprising contacting a candidate compound with a INSP181 gene or polypeptide, and selecting compounds that bind said gene or polypeptide.

A further other object of this invention resides in methods of selecting biologically active compounds, said method comprising contacting a candidate compound with recombinant host cell expressing a INSP181 polypeptide with a candidate compound, and selecting compounds that bind said INSP181 polypeptide at the surface of said cells and/or that modulate the activity of the INSP181 polypeptide.

A “biologically active” compound denotes any compound having biological activity in a subject, preferably therapeutic activity, more preferably a compound having lipocalin activity, and further preferably a compound that can be used for treating INSP181 related disorders, or as a lead to develop drugs for treating lipocalin related disorder. A “biologically active” compound preferably is a compound that modulates the activity of INSP181.

The above methods may be conducted in vitro, using various devices and conditions, including with immobilized reagents, and may further comprise an additional step of assaying the activity of the selected compounds in a model of lipocalin related disorder, such as an animal model.

Preferred selected compounds are agonists of INSP181, i.e., compounds that can bind to INSP181 and mimic the activity of an endogenous ligand thereof.

A further object of this invention resides in a method of selecting biologically active compounds, said method comprising contacting in vitro a test compound with a INSP181 polypeptide according to the present invention and determining the ability of said test compound to modulate the activity of said INSP181 polypeptide.

A further object of this invention resides in a method of selecting biologically active compounds, said method comprising contacting in vitro a test compound with a INSP181 gene according to the present invention and determining the ability of said test compound to modulate the expression of said INSP181 gene, preferably to stimulate expression thereof.

In another embodiment, this invention relates to a method of screening, selecting or identifying active compounds, particularly compounds active on multiple sclerosis or related disorders, the method comprising contacting a test compound with a recombinant host cell comprising a reporter construct, said reporter construct comprising a reporter gene under the control of a INSP181 gene promoter, and selecting the test compounds that modulate (e.g. stimulate or reduce, preferably stimulate) expression of the reporter gene.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Binding to a target gene or polypeptide provides an indication as to the ability of the compound to modulate the activity of said target, and thus to affect a pathway leading to lipocalin related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

Modulation of activity includes, without limitation, stimulation of the surface expression of the INSP181 receptor, modulation of multimerization of said receptor (e.g., the formation of multimeric complexes with other sub-units), etc. The cells used in the assays may be any recombinant cell (i.e., any cell comprising a recombinant nucleic acid encoding a INSP181 polypeptide) or any cell that expresses an endogenous INSP181 polypeptide. Examples of such cells include, without limitation, prokaryotic cells (such as bacteria) and eukaryotic cells (such as yeast cells, mammalian cells, insect cells, plant cells, etc.). Specific examples include E. coli, Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing (optionally on the surface thereof) the polypeptide according to the first aspect of the invention, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

Methods for generating detectable signals in the types of assays described herein will be known to those of skill in the art. A particular example is cotransfecting a construct expressing a polypeptide according to the invention, or a fragment such as the LBD, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential ligand is added to the cells, it will bind the GAL4-polypeptide fusion and induce transcription of the luciferase gene. The level of the luciferase expression can be monitored by its activity using a luminescence reader (see, for example, Lehman et al. JBC 270, 12953, 1995; Pawar et al. JBC, 277, 39243, 2002).

A further preferred method for identifying an agonist or antagonist of a polypeptide of the invention comprises:

(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, by means of artificially anchoring it to the cell membrane, or by constructing a chimeric receptor being associated with a second component capable of providing a detectable signal in response to the binding of a compound to the polypeptide, with a compound to be screened under conditions to permit binding to the polypeptide; and
(c) determining whether the compound binds to and activates or inhibits the polypeptide by comparing the level of a signal generated from the interaction of the compound with the polypeptide with the level of a signal in the absence of the compound.

For example, a method such as FRET detection of ligand bound to the polypeptide in the presence of peptide co-activators (Norris et al, Science 285, 744, 1999) might be used.

A further preferred method for identifying an agonist or antagonist of a polypeptide of the invention comprises:

(a) contacting a cell expressing (optionally on the surface thereof) the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to cells which express a polypeptide of the invention (and which optionally have a polypeptide of the invention on the surface thereof), or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention, optionally on the cell surface, or a cell membrane containing a polypeptide of the invention,
(b) measuring the amount of labelled ligand bound to the whole cell or the cell membrane;
(c) adding a candidate compound to a mixture of labelled ligand and the whole cell or the cell membrane of step (a) and allowing the mixture to attain equilibrium;
(d) measuring the amount of labelled ligand bound to the whole cell or the cell membrane after step (c); and
(e) comparing the difference in the labelled ligand bound in step (b) and (d), such that the compound which causes the reduction in binding in step (d) is considered to be an agonist or antagonist.

Similarly, there is provided a method of screening for a polypeptide antagonist or agonist compound which comprises the steps of:

(a) incubating a labelled ligand with a polypeptide according to the invention on any solid support or the cell surface, or a cell membrane containing a polypeptide of the invention.
(b) measuring the amount of labelled ligand bound to the polypeptide on the solid support, whole cell or the cell membrane;
(c) adding a candidate compound to a mixture of labelled ligand and immobilized polypeptide on the solid support, the whole cell or the cell membrane of step (a) and allowing the mixture to attain equilibrium;
(d) measuring the amount of labelled ligand bound to the immobilized polypeptide or the whole cell or the cell membrane after step (c); and
(e) comparing the difference in the labelled ligand bound in step (b) and (d), such that the compound which causes the reduction in binding in step (d) is considered to be an agonist or antagonist.

The INSP181 polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signalling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptides of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

In another embodiment, this invention relates to the use of a INSP181 polypeptide or fragment thereof, whereby the fragment is preferably a INSP181 gene-specific fragment, for isolating or generating an agonist or stimulator of the INSP181 polypeptide for the treatment of an immune related disorder, wherein said agonist or stimulator is selected from the group consisting of:
1. a specific antibody or fragment thereof including:
   a) a chimeric,
   b) a humanized or
   c) a fully human antibody, as well as;
2. a bispecific or multispecific antibody,
3. a single chain (e.g. scFv) or
4. single domain antibody, or
5. a peptide- or non-peptide mimetic derived from said antibodies or
6. an antibody-mimetic such as a) an anticalin or b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

As mentioned above, it is envisaged that the various moieties of the invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may be useful in the therapy or diagnosis of diseases. To assess the utility of the moieties of the invention for treating or diagnosing a disease one or more of the following assays may be carried out. Note that although some of the following assays refer to the test compound as being a protein/polypeptide, a person skilled in the art will readily be able to adapt the following assays so that the other moieties of the invention may also be used as the “test compound”.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98%. 98.5% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thyrnine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly certain diseases including, but not limited to, vision disorders (e.g. nightblindness), immune system disorders (e.g. autoimmune disorders), inflammatory disorders, inflammatory bowel disease (IBD), ulcerative colitis (UC), Crohn's disease (CD), proctitis, cell proliferative disorders, cancer (e.g. breast cancer), microbial infections (e.g. viral, bacterial and fungal infections), emphysema, skin diseases, reproductive disorders (e.g. infertility, in particular male infertility), renal dysfunction, myocardial infarction, arthritis, and multiple sclerosis, gross cystic breast disease and regulation of nervous system development.

Various aspects and embodiments of the present invention will now be described in more detail by way of example, with particular reference to INSP181 polypeptides.

It will be appreciated that modification of detail may be made without departing from the scope of the invention.

### BRIEF DESCRIPTION OF THE FIGURES

FIG. 1: BLAST results for INSP181 versus public protein database.

FIG. 2: Alignment of top blast hit against INSP181.

FIG. 3: SignalP output for INSP181.

FIG. 4: Multiple sequence alignment of INSP181 and related Lipocalin domain containing sequences.

FIG. 5: INSP181 DNA and protein sequence. The position and sense of the PCR primers are indicated by arrows.

FIG. 6: The nucleotide sequence alignment of cDNAs cloned using INSP181-CP3 and INSP181-CP4 PCR primers with the INSP181 prediction.

FIG. 7: The amino acid sequence alignment of cDNAs cloned using INSP181-CP3 and INSP181-CP4 PCR primers with the INSP181 prediction.

FIG. 8: Nucleotide sequence with translation of the INSP181 PCR product cloned using primers INSP181-CP3 and INSP181-CP4.

FIG. 9: Nucleotide sequence with translation of the INSP181-SV PCR product cloned using primers INSP181-CP3 and INSP181-CP4.

FIG. 10: NetNGyc results for INSP181. A glycosylation site is indicated at position 92.

FIG. 11: Sequence Translation and Features of INSP181.

FIG. 12: Expression of INSP181 in major human tissues as measured by RT-PCR (TaqMan).

FIG. 13: Expression of INSP181 in diseased skin biopsies from IL18BP clinical trial as measured by RT-PCR (TaqMan).

FIG. 14: Domain Professor information showing the predicated lipocalin domain in INSP181.

FIG. 15: Family/Residue Information for the Lipocalin domain showing secondary structure predictions and the location of the disulphide bridge.

TABLE 1

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 2

| Amino | |
|---|---|
| Acid | Synonymous Groups |
| Ser | D-Ser, Thr, D-Thr, allo-Thr, Met, D-Met, Met(O), D-Met(O), L- |
| | Cys, D-Cys |
| Arg | D-Arg, Lys, D-Lys, homo-Arg, D-homo-Arg, Met, Ile, D-.Met, |
| | D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or |
| | L-1-oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, or |
| | 5-phenylproline, Bpa, D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, |
| | D-Met, Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S--Me--Cys, Ile, D-Ile, Leu, D-Leu, Val, D-Val |

### EXAMPLES

### Example 1

### INSP181

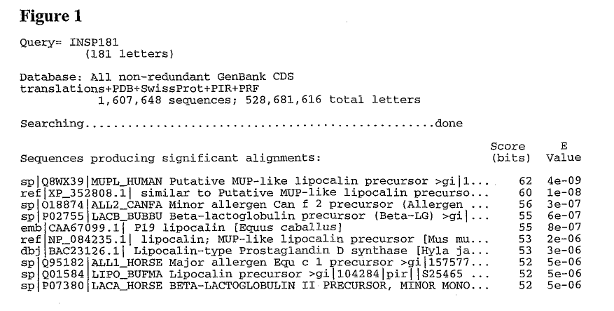

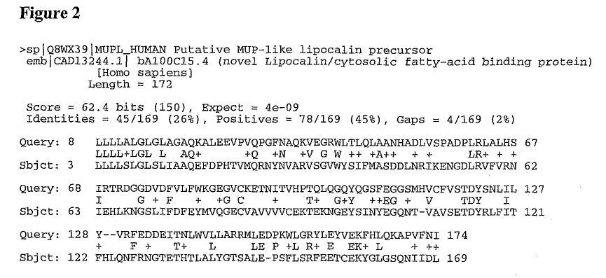

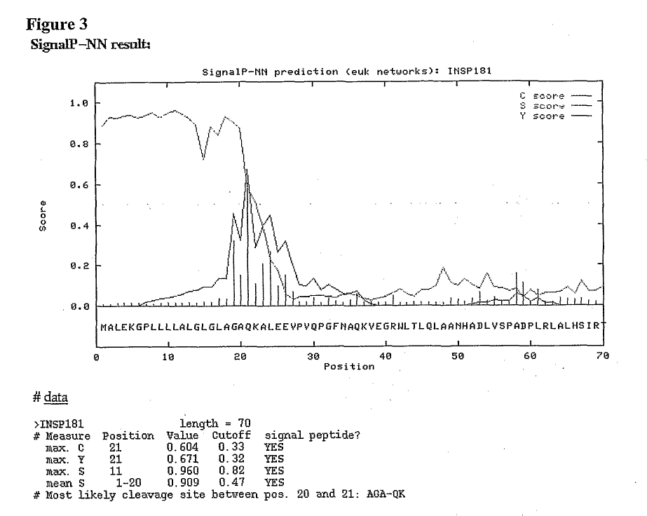

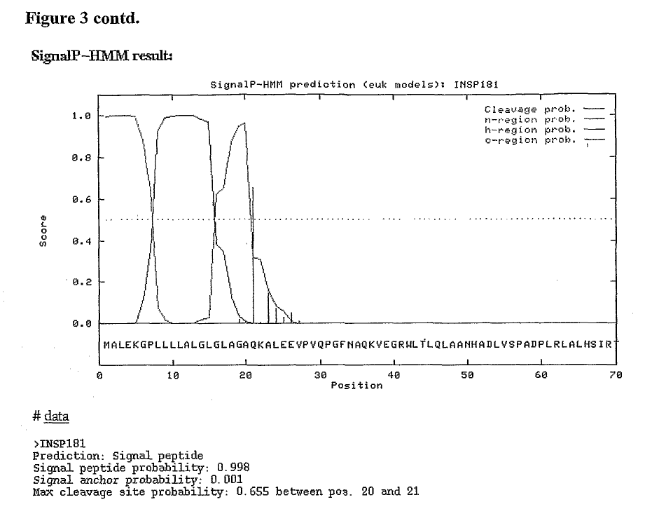

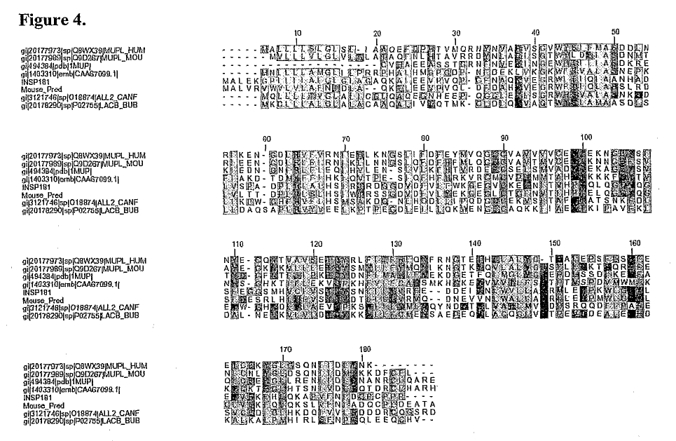

The INSP181 polypeptide sequence was used as a BLAST query against the NCBI non-redundant sequence database. The top ten matches from the BLAST query are shown in FIG. 1. FIG. 2 shows the alignment of the INSP181 query sequence to the top blast hit.

The cloning of the INSP181 cDNA will allow expression of the INSP181 protein in prokaryotic or eukaryotic expression systems and its subsequent purification and characterisation. For example, recombinant INSP181 may be used to generate INSP181-specific monoclonal or polyclonal antibodies which might then be used in the biochemical characterisation of INSP181. Alternatively, recombinant INSP181 may be used in a wide variety of screening assays, including those described above, and those described in Example 5 below.

### Example 2

### Cloning of INSP181 and INSP181 SV

2.1 Preparation of Human cDNA Templates

First strand cDNA was prepared from a variety of human tissue total RNA samples (Clontech, Stratagene, Ambion, Biochain Institute and in-house preparations) using Superscript II or SuperScript III RNase H⁻ Reverse Transcriptase (Invitrogen) according to the manufacturer's protocol.

For SuperScript II: Oligo (dT)₁₅ primer (1 μl at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and chilled on ice. The contents were collected by brief centrifugation and 4 μl of 5× First-Strand Buffer, 2 μl 0.1 M DTT, and 1 μl RnaseOUT™ Recombinant Ribonuclease Inhibitor (40 units/μl, Invitrogen) were added. The contents of the tube were mixed gently and incubated at 42° C. for 2 min, then 1 μl (200 units) of SuperScript II™ enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min.

For SuperScript III: 1 μl Oligo(dT)₂₀ primer (50 μM, Invitrogen), 2 μg human total RNA, 1 μl 10 nM dNTP mix (10 mM each of dATP, dGTP, dCTP and dTTP at neutral pH) and sterile distilled water to a final volume of 10 μl were combined in a 1.5 ml Eppendorf tube, heated to 65° C. for 5 min and then chilled on ice. For each RT reaction a cDNA synthesis mix was prepared as follows: 2 μl 10×RT buffer, 4 μl 25 mM MgCl₂, 2 μl 0.1 M DTT, 1 μl RNaseOUT™ (40 U/μl) and 1 μl SuperScript III™ RT enzyme were combined in a separate tube and then 10 μl of this mix added to the tube containing the RNA/primer mixture. The contents of the tube were mixed gently, collected by brief centrifugation, and incubated at 50° C. for 50 min. The reaction was terminated by incubating at 80° C. for 5 min and the reaction mixture then chilled on ice and collected by brief centrifugation. To remove RNA complementary to the cDNA, 1 μl (2 units) of E. coli RNase H (Invitrogen) was added and the reaction mixture incubated at 37° C. for 20 min.

The final 21 μl reaction mix was diluted by adding 179 μl sterile water to give a total volume of 200 μl. The RNA samples were combined into pools such that each pool contained up to five different cDNA samples. 5 μl of each cDNA pool was used as a template for PCR in a 50 μl final reaction volume and this consisted of 1 μl of each cDNA sample in that pool. This represented approximately 20 ng of each individual cDNA template.

2.2 cDNA Libraries

Human cDNA libraries (in bacteriophage lambda (λ) vectors) were purchased from Stratagene, Clontech or Invitrogen, or prepared at the Serono Pharmaceutical Research Institute in λ ZAP, λ GT10, λ GT11, or TriplEx2 vectors according to the manufacturer's protocol (Stratagene and Clontech). Bacteriophage λ DNA was prepared from small scale cultures of infected E. coli host strain using the Wizard Lambda Preps DNA purification system according to the manufacturer's instructions (Promega, Corporation, Madison Wis.).

### 2.3 Gene Specific Cloning Primers for PCR

Two pairs of PCR primers having a length of between 18 and 30 bases were designed for amplifying the INSP181 predicted cds using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP181) with little or no none specific priming. The primers were designed to form two nested pairs such that INSP181-CP3/INSP181-CP4 primers were positioned internally to primers INSP181-CP1/INSP181-CP2.

2.4 PCR Amplification of INSP181 from Human cDNA Templates

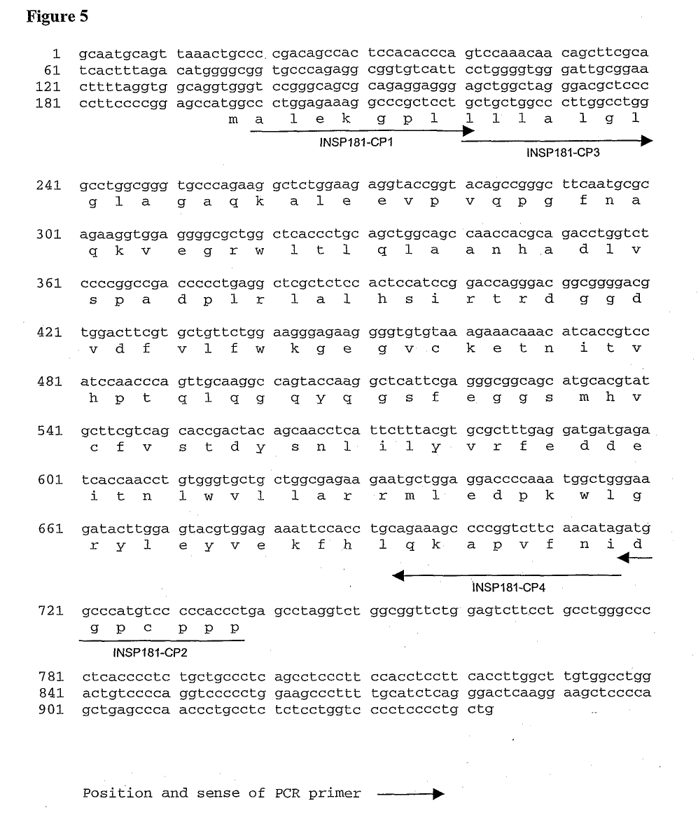

Gene-specific cloning primers INSP181-CP1 and INSP181-CP2 (Table 1, FIG. 5) were designed to amplify a cDNA fragment of 540 bp containing the INSP181 cds. The primer pair was used with the panel of cDNA libraries and pools of human cDNA samples as PCR templates. This PCR1 was performed in a final volume of 50 μl containing 1× AmpliTaq™ buffer, 200 μM dNTPs, 50 pmoles of each cloning primer, 2.5 units of AmpliTaq™ (Applied Biosystems) and approximately 20 ng of cDNA library template or 100 ng cDNA pool template using an MJ Research DNA Engine, programmed as follows: 94° C., 2 min; 40 cycles of 94° C., 1 min, 55° C., 1 min, and 72° C., 1 min; followed by 1 cycle at 72° C. for 7 min and a holding cycle at 4° C.

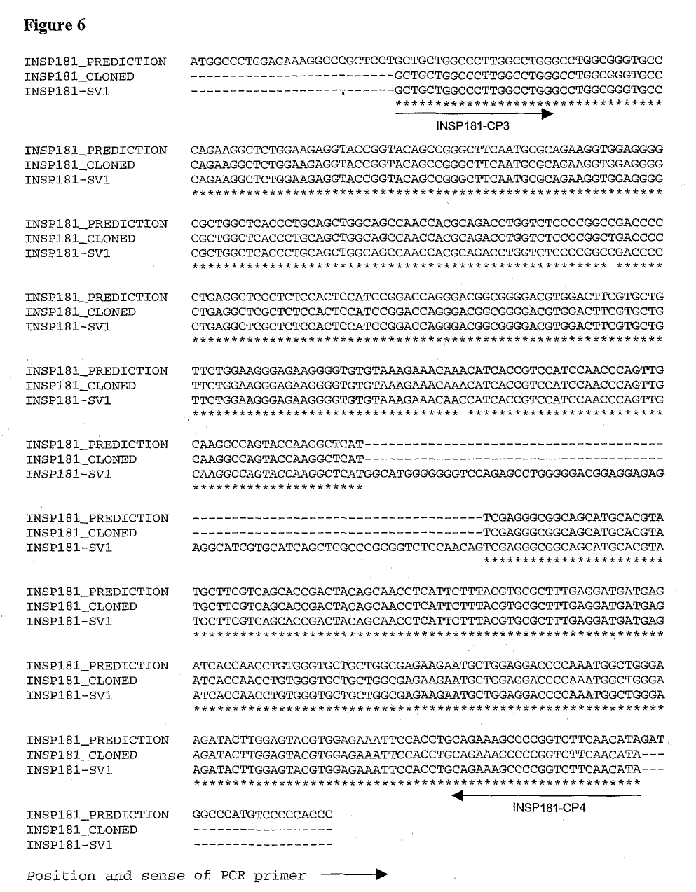

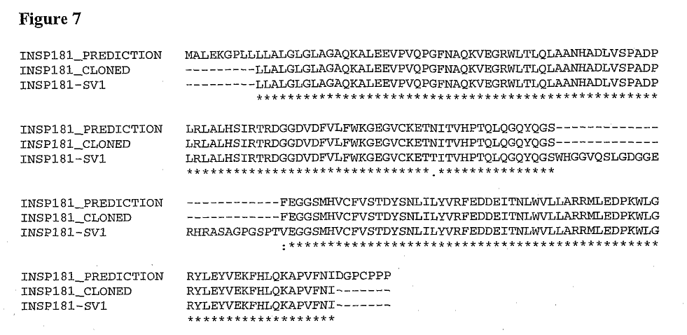

Each PCR1 product was then used as the template for PCR2 using amplification primers INSP181-CP3 and INSP181-CP4 (Table 1, FIG. 5-9) designed to amplify a cDNA fragment of 496 bp within the INSP181-CP1/INSP181-CP2 product. PCR2 was performed in a final volume of 50 μl containing 1× AmpliTaq™ buffer, 200 μM dNTPs, 50 pmoles of each cloning primer, 2.5 units of AmpliTaq™ (Applied Biosystems), and 1 μl of PCR1 product using an MJ Research DNA Engine, programmed as follows: 94° C., 2 min; 40 cycles of 94° C., 1 min, 59° C., 1 min, and 72° C., 1 min; followed by 1 cycle at 72° C. for 7 min and a holding cycle at 4° C.

30 μl of each PCR1 and PCR2 amplification product was visualized on a 0.8% agarose gel in 1×TAE buffer (Invitrogen). Products of the approximately the expected molecular weight (540 bp for PCR1, 496 bp for PCR2) were purified from the gel using the Wizard PCR Preps DNA Purification System (Promega), eluted in 50 μl of water and subcloned directly.

### 2.5 Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

### 2.6 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Applied Biosystems) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR products were analyzed on 1% agarose gels in 1×TAE buffer. Colonies which gave PCR products of approximately the expected molecular weight (540 bp or 496 bp+105 bp due to the multiple cloning site (MCS)) were grown up overnight at 37° C. in 5 ml L-Broth (LB) containing ampicillin (100 μg/ml), with shaking at 220 rpm.

### 2.7 Plasmid DNA Preparation and Sequencing

Miniprep plasmid DNA was prepared from the 5 ml cultures using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Plasmid DNA (200-500 ng) was subjected to DNA sequencing with the sequencing primers T7 and T3 (Table 1) using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analyzed on an Applied Biosystems 3700 sequencer.

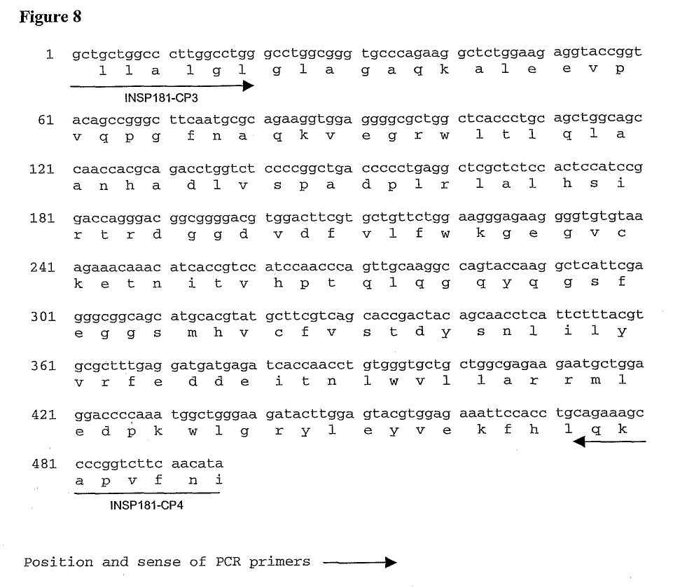

Sequence analysis identified a clone, amplified from a pool containing cDNA derived from an atherosclerotic plaque and basophils in PCR2, which contained the expected INSP181-CP3/INSP181-CP4 PCR product sequence. The sequence of the cloned cDNA fragment is shown in FIG. 8. The cloned PCR product is in plasmid pCR4-TOPO-INSP181.

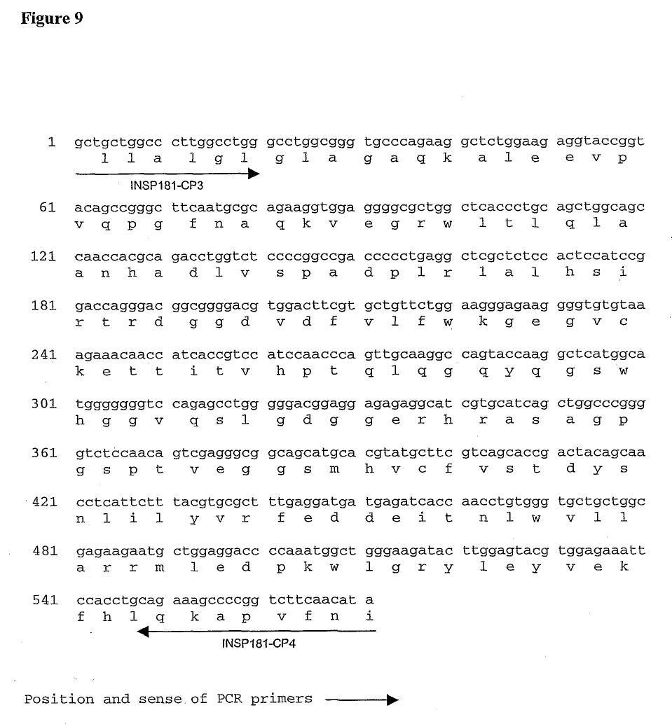

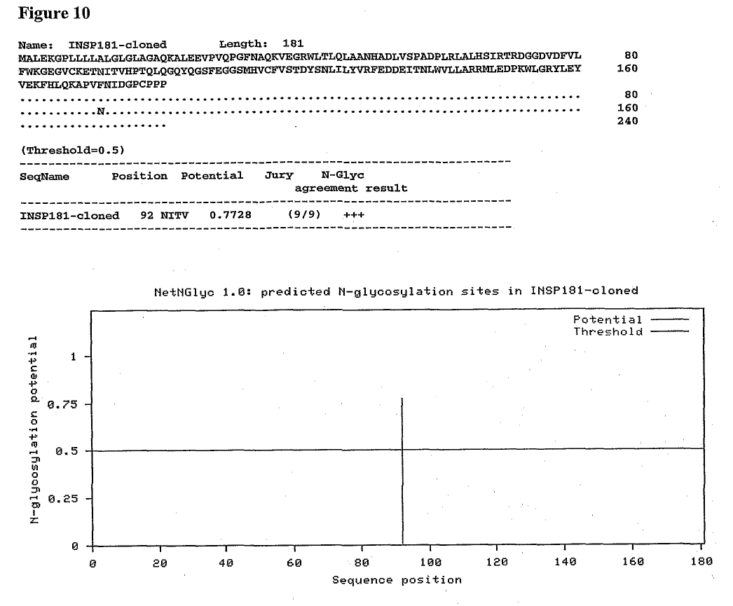

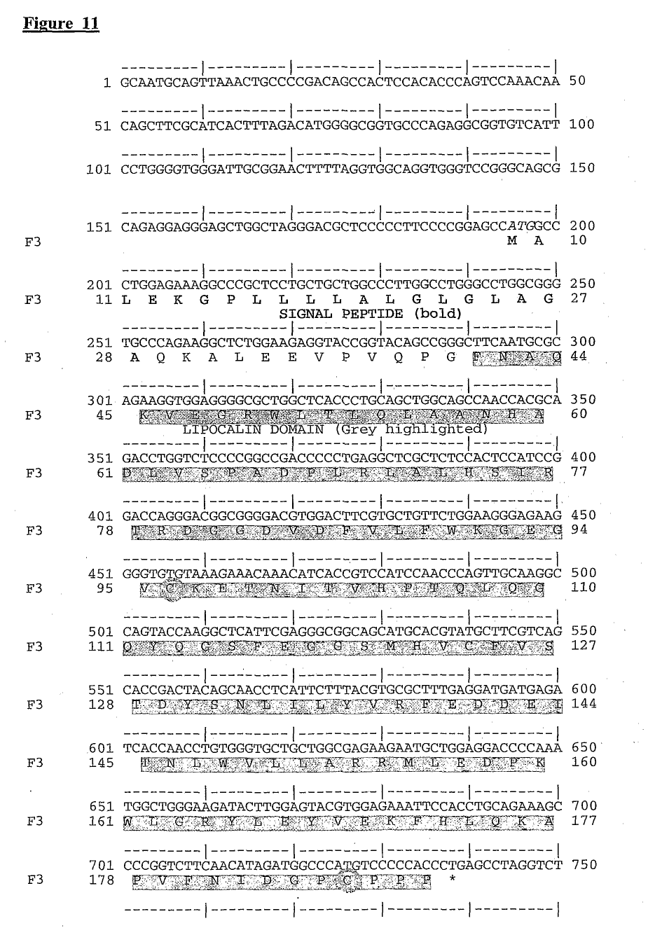

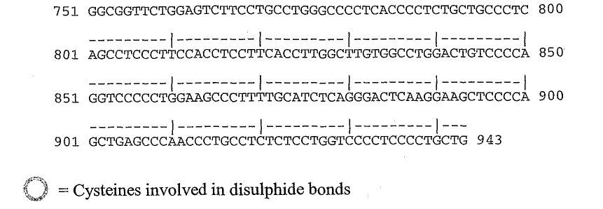

A second clone was identified, amplified from a pool containing cDNA derived from salivary gland, adrenal gland, eye, and the Stratagene universal reference RNA template in PCR2, which contained the expected INSP181-CP3/INSP181-CP4 product, but with a 25 amino acid insertion at the start of exon 4. This insertion led to the amino acid substitution F113V. On comparison with genomic DNA sequence, the clone also contained the amino acid substitutions N92T and G114S which may be PCR-induced errors although polymorphisms cannot be ruled out at this stage. The sequence of the cloned cDNA fragment is shown in FIG. 9. The cloned PCR product is in plasmid pCR4-TOPO-INSP181-SV1.

TABLE 1

| | |
|---|---|
| Primer | Sequence (5′-3′) |

| | |
|---|---|
| INSP181-CP1 | CCC TGG AGA AAG GCC CGC TCC TG |
| INSP181-CP2 | AGG GTG GGG GAC ATG GGC CAT C |
| INSP181-CP3 | GCT GCT GGC CCT TGG CCT GG |
| INSP181-CP4 | TAT GTT GAA GAC CGG GGC TTT CTG |
| T7 | TAA TAC GAC TCA CTA TAG G |
| T3 | ATT AAC CCT CAC TAA AGG |

### Example 3

### Generation of Gateway Compatible INSP1810RF Fused to an in Frame 6HIS Tag Sequence

INSP181 was cloned by nested PCR and therefore the cDNA insert in the pCR4-TOPO clone (plasmid pCR4-TOPO-INSP181) was missing 26 bp at the 5′ end and 21 bp at the 3′ end of the coding sequence. Incorporation of missing nucleotides, 6HIS tag and stop codon were all accomplished by including the appropriate nucleotides in the primers used for PCR amplification. The sequences of PCR primers used in the cloning are shown in table 2.

TABLE 2

| | |
|---|---|
| Primer | Sequence (5′-3′) |

| | |
|---|---|
| INSP181 | GCC ACC ATG GCC CTG GAG AAA GGC CCG CTC |
| MAT FP | CTG CTG CTG GCC CTT GGC |
| INSP181 | GTG ATG GTG ATG GTG GGG TGG GGG ACA TGG |
| MAT RP | GCC ATC GTT GAA GAC CGG |
| INSP181 | G GGG ACA AGT TTG TAC AAA AAA GCA GGC |
| ATTB1 FP | TTC GCC ACC ATG GCC CTG |
| ATTB1 PCR | GGG GAC CAC TTT GTA CAA GAA AGC TGG GTT |
| RP | TCA ATG GTG ATG GTG ATG GTG |
| INSP181 SV1 | GGG TGT GTA AAG AAA CAA ACA TCA CCG TCC |
| (T92N) FP | ATC CAA C |
| INSP181 SV1 | GTT GGA TGG ACG GTG ATG T TTG TTT CTT |
| (T92N) RP | TAC ACA CCC |
| INSP181SV1 | TGG CAT GGG GGG GTC CAG G GCC TGG GGG |
| (S114G) FP | ACG GAG GAG |
| INSP181SV1 | CTC CTC CGT CCC CCA GGC C CTG GAC CCC |
| (S114G) RP | CCC ATG CCA |
| 21 M13 FP | CGA CGT TGT AAA ACG ACG GCC AGT |
| M13 RP | CAG GAA ACA GCT ATG AC |
| pEAK12 FP | AGC CTC AGA CAG TGG TTC AA |
| pEAK12 RP | GAT GGA GGT GGA CGT GTC AG |

Underlined sequence = Kozak sequence
Bold = Stop codon
Italic sequence = His tag

Plasmid pCR4-TOPO-INSP181 was used as PCR template to generate the full-length ORF containing a C-terminal 6HIS tag and a stop codon. The first stage of this Gateway cloning process involved a two step PCR reaction which generates the full-length ORF of INSP181 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction PCR1, (in a final volume of 50 μl) contains respectively: 1 μl (25 ng) of plasmid pCR4-TOPO-INSP181, 4.0 μl dNTPs (10 mM), 5 μl of 10×Pfx polymerase buffer, 1 μl MgSO4 (50 mM), 1.0 μl each of gene specific primer (to give a final concentration of 100 pmoles) (INSP181 MAT FP and INSP181 MAT RP), and 0.5 μl Platinum Pfx DNA polymerase (Invitrogen). The PCR reaction was performed using an initial denaturing step of 95° C. for 2 min, followed by 30 cycles of 94° C. for 30 s; 64° C. for 30 s and 68° C. for 1 min; and a final extension cycle of 68° C. for 5 minutes and a holding cycle of 4° C. The amplification product was directly purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (543 bp+24 bp=567 bp)

The second PCR reaction (in a final volume of 50 μl) contained 1 μl of diluted purified PCR1 product (to a final concentration of 10 ng), 4.0 μl dNTPs (10 mM), 5 μl of 10×Pfx polymerase buffer, 1 μl MgSO₄ (50 mM), 1.0 μl of each Gateway conversion primer (to give a final concentration of 100 pmoles) (INSP181 ATTB1 FP and ATTB1 PCR RP) and 0.5 μl of Platinum Pfx DNA polymerase. The conditions for the 2nd PCR reaction were: 95° C. for 2 min, followed by 30 cycles of 94° C. for 30 s; 60° C. for 30 s and 68° C. for 1 min; and a final extension cycle of 68° C. for 5 minutes and a holding cycle of 4° C. The PCR product was gel purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (567 bp+64 bp=631 bp)

3.1 Subcloning of Gateway Compatible INSP181-6HIS ORF into Gateway Entry Vector pDONR221

The second stage of the Gateway cloning process involved subcloning of the Gateway modified PCR product into the Gateway entry vector pDONR221 (Invitrogen) as follows: 5 μl of gel extracted product from PCR2 was incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. An aliquot of this reaction (2 μl) was used to transform DH5a strain (Invitrogen) as follows: a 50 μl aliquot of DH5a cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (250 rpm) for 1 h at 37° C. The transformation mixture was then plated on L-broth (LB) plates containing kanamycin (40 μg/ml) and incubated overnight at 37° C.

Five transformants were picked and patched on LB agar plates containing kanamycin (40 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 picomoles) (21M13 FP and ATTB1 PCR RP) and 0.5 μl of Taq DNA polymerase.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from 5 ml cultures using QIAprep Spin Miniprep kit (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 2. Sequencing reactions were analyzed on CEQ 2000 XL DNA analysis system (Beckman Coulter P/N 608450). After sequence confirmation of the insert, pDONR221_INSP181-6HIS was then used for creating the expression clones.

3.2 Subcloning of Gateway compatible INSP181 ORF into Expression Vectors pEAK12d and pDEST12.2

Plasmid DNA (2 μl or approx. 150 ng) of pDONR221_INSP181-6HIS was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl.

The reaction was stopped by addition of 1 μl proteinase K (2 μg/μl) and incubated at 37° C. for a further 10 min. An aliquot of this reaction (2 l) was used to transform DH5a strain (Invitrogen) as follows: a 50 μl aliquot of DH5αα cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (250 rpm) for 1 h at 37° C. The transformation mixture was then plated on L-broth (LB) plates containing Ampicillin (100 μg/ml) and incubated overnight at 37° C.

Five transformants were picked and patched on LB agar plates containing Ampicillin (100 μg/ml) and incubated overnight at 37° C. A scoop of the grown culture from the patched plate was resuspended in 50 ul of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 picomoles and 0.5 μl of Taq DNA polymerase. pEAK12d clones were screened using the primers pEAK12 FP and INSP181 MAT RP and pDEST12.2 clones were screened using the primers 21M13FP and INSP181 MAT RP.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from 5 ml cultures using QIAprep Spin Miniprep kit (Qiagen).

Plasmid DNA (150-200 ng) in the pEAK12d vector was subjected to DNA sequencing with the sequencing primers pEAK12 FP and pEAK12 RP as described above. Plasmid DNA (150-200 ng) in the pDEST12.2 vector was subjected to DNA sequencing with the sequencing primers 21M13 FP and M13Rev RP as described above.

Sequence confirmed clones were designated as pEAK12d_INSP181-6HIS and pDEST12.2_INSP181-6HIS.

Maxi-prep DNA was prepared from a 500 ml culture of the sequence verified clone pEAK12d_INSP181-6HIS using a Qiagen Maxi prep kit according to the manufacturer's instructions. Plasmid DNA was resuspended at a concentration of 1 μg/μl in TE buffer and stored at −20° C.

Endotoxin-free maxi-prep DNA was prepared from a 500 ml culture of the sequence verified clone (pDEST12.2_INSP181-6HIS) using the EndoFree Plasmid Mega kit (Qiagen) according to the manufacturer's instructions. Purified plasmid DNA was resuspended in endotoxin free TE buffer at a final concentration of at least 3 μg/μl and stored at −20° C.

### Example 4

### Generation of Gateway Compatible INSP181 SV10RF Fused to an in Frame 6HIS Tag Sequence

INSP181 SV1 was cloned by nested PCR and therefore the cDNA insert in the pCR4-TOPO clone (plasmid pCR4-TOPO-INSP181-SV1) was missing 26 bp at the 5′ end and 21 bp at the 3′ end of the coding sequence. Also, two mutations resulting in the amino acid changes (N92T and G114S) were detected on sequencing which needed to be corrected. Incorporation of missing nucleotides, 6HIS tag and stop codon were all accomplished by including the appropriate nucleotides in the primers used for PCR amplification. Site directed mutagenesis was carried out to correct the two mutations after the full-length INSP181 SV1 entry clone was created.

Plasmid pCR4-TOPO-INSP181-SV1 was used as PCR template to generate the full-length ORF containing a C-terminal 6HIS tag and a stop codon. The first stage of this Gateway cloning process involved a two step PCR reaction which generates the full-length ORF of INSP181SV1 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction PCR1, (in a final volume of 50 μl) contains respectively: 1 μl (25 ng) of plasmid pCR4-TOPO-INSP181-SV1, 4.0 μl dNTPs (10 mM), 5 μl of 10×Pfx polymerase buffer, 1 μl MgSO4 (50 mM), 1.0 μl each of gene specific primer (to give a final concentration of 100 pmoles) (INSP181 MAT FP and INSP181 MAT RP), and 0.5 μl Platinum Pfx DNA polymerase (Invitrogen). The PCR reaction was performed using an initial denaturing step of 95° C. for 2 min, followed by 30 cycles of 94° C. for 30 s; 64° C. for 30 s and 68° C. for 1 min; and a final extension cycle of 68° C. for 5 minutes and a holding cycle of 4° C. The amplification product was directly purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (618 bp+24 bp=642 bp).

The second PCR reaction (in a final volume of 50 μl) contained 1 μl of diluted purified PCR1 product (to a final concentration of 10 ng), 4.0 μl dNTPs (10 mM), 5 μl of 10×Pfx polymerase buffer, 1 μl MgSO₄ (50 mM), 1.0 μl of each Gateway conversion primer (to give a final concentration of 100 pmoles) (INSP181 ATTB1 FP and ATTB1 PCR RP) and 0.5 μl of Platinum Pfx DNA polymerase. The conditions for the 2nd PCR reaction were: 95° C. for 2 min, followed by 30 cycles of 94° C. for 30 s; 60° C. for 30 s and 68° C. for 1 min; and a final extension cycle of 68° C. for 5 minutes and a holding cycle of 4° C. The PCR product was gel purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (642 bp+64 bp 706 bp)

4.1 Subcloning of Gateway Compatible INSP181SV1-6HIS ORF into Gateway Entry Vector pDONR221

The second stage of the Gateway cloning process involved sub cloning of the Gateway modified PCR product into the Gateway entry vector pDONR221 (Invitrogen) as follows: 5 μl of gel extracted product from PCR2 was incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/□l) and incubated at 37° C. for a further 10 min. An aliquot of this reaction (2 μl) was used to transform DH5α strain (Invitrogen) as follows: a 50 μl aliquot of DH5a cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (250 rpm) for 1 h at 37° C. The transformation mixture was then plated on L-broth (LB) plates containing kanamycin (40 μg/ml) and incubated overnight at 37° C.

Five transformants were picked and patched on LB agar plates containing kanamycin (40 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 pmoles) (21M13 FP and ATTB1 PCR RP) and 0.5 μl of Taq DNA polymerase.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from 5 ml cultures using QIAprep Spin Miniprep kit (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 2. Sequencing reactions were analyzed on CEQ 2000 XL DNA analysis system (Beckman Coulter P/N 608450). After sequence confirmation, pDONR221_INSP181SV1 (N92T, G114S)-6HIS was used as a template for site directed mutagenesis to correct the two mutations.

### 4.2 Site Directed Mutagenesis of INSP181SV1-6HIS

The INSP181SV1 sequence cloned by PCR differed from the predicted INSP181SV1 by substitution at two different positions (A 275 C and G 340 A) which leads to amino acid mutations N 92 T and G 114 S. These mutations were thought to result from the PCR cloning procedure as they were not detected in genomic DNA (Celera or Genbank). In order to create a pDONR221 clone containing the correct INSP181SV1 sequence, the pDONR221_INSP181SV1-(N92T, G114S)-6HIS clone was used as a template for site-directed mutagenesis.

### 4.3 Gene Specific Cloning Primers for Site-Directed Mutagenesis of INSP181SV1

Two pairs of PCR primers, INSP181SV1 (T92N) FP, INSP181SV1 (T92N) RP and INSP181SV1 (S114G) FP and INSP181SV1 (S114G) RP (Table 2), were designed such that the primers annealed to opposite strands of the pDON221_INSP181SV1-(N92T, G114S)-6HIS sequence and each primer annealed to 15-25 bases on either side of the amino acid to be mutated. The PCR primers were designed following the instructions given in the Instruction manual for the QuickChange® II XL Site-Directed Mutagenesis Kit (Stratagene).

### 4.4 Site-Direct Mutagenesis of INSP181SV1

Site-directed mutagenesis-1, was carried out using the QuickChange® II Site-Directed Mutagenesis Kit (Stratagene) according to the manufacturer's instructions. The control reaction was performed in a final volume of 50 μl containing 1× reaction buffer, 10 ng pWhitescript 4.5 kb control plasmid, 125 ng oligonucleotide control primer #1, 125 ng control primer #2, 1 μl dNTP mix, and 2.5 units PfuUltra HF DNA polymerase. The sample reaction was performed in a final volume of 50 μl containing 1× reaction buffer, 10 ng plasmid template DNA (pDONR221_INSP181SV1-(N92T, G114S)-6HIS), 125 ng INSP181SV1 (T92N) FP, 125 ng INSP181SV1 (T92N) RP, 1 μl dNTP mix, and 2.5 units PfuUltra HF DNA polymerase. Thermal cycling was performed using a MJ Research DNA Engine, programmed as follows: 95° C., 1 min; 18 cycles of 95° C., 30 sec, 60° C., 1 min, and 68° C., 3 min 30 sec; followed by a final extension of 68° C. for 7 min and a holding cycle at 4° C.

Dpn I digestion was used to digest the methylated or hemimethylated parental DNA template (plasmid pDONR221_INSP181SV1-6HIS in the sample reaction). 1 μl of Dpn I restriction enzyme (10 U/μl, Stratagene) was added to the products of the control and sample amplification reactions. The reactions were mixed gently and incubated at 37° C. for 1 hour. Each reaction mixture was then transformed into XL1-Blue supercompetent cells (Stratagene) as follows. A 50 μl aliquot of XL1-Blue cells was thawed on ice and 1 μl of Dpn I-treated DNA was added. The mixture was incubated for 30 min on ice and then heat shocked by incubation at 42° C. for exactly 45 s. Samples were returned to ice for 2 min and 250 μl of pre-warmed (42° C.) NZY media was added. Samples were incubated with shaking (220 rpm) for 1 h at 37° C. The control transformation mixture (250 μl) was then plated on an L-broth (LB) plate containing Ampicillin (100 μg/ml), X-gal (80 μg/ml), and 20 mM IPTG. The sample transformation mixture (250 μl on each of 2 plates) was plated on L-broth (LB) plates containing Kanamycin (40 μg/ml). Plates were incubated overnight at 37° C.

### 4.5 Plasmid DNA Preparation and Sequencing

One transformant was selected and plasmid mini-prep DNA was prepared from a 5 ml culture using QIAprep Spin Miniprep kit (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 2. Sequencing reactions were analyzed on CEQ 2000 XL DNA analysis system (Beckman Coulter P/N 608450). After sequence confirmation of the insert with the corrected mutation, pDONR221_INSP181SV1-(G114S)-6HIS was then used as a template for making the second correction (S114G). Site directed mutagenesis-2, was carried out using the protocol and conditions mentioned above. The primers used for site directed mutagenesis-2 were INSP181SV1 (S114G) FP and INSP181SV1 (S114G) RP. Sequence analysis identified a clone which contained the expected INSP181SV1 insert sequence (pDONR221_INSP181SV1-6HIS).

4.6 Subcloning of Gateway Compatible INSP181SV10RF into Expression Vectors pEAK12d and pDEST12.2

Plasmid DNA (2 μl or approx. 150 ng) of pDONR221_INSP181SV1-6HIS was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl.

The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (250 rpm) for 1 h at 37° C. The transformation mixture was then plated on L-broth (LB) plates containing ampicillin (100 μg/ml) and incubated overnight at 37° C.

Five transformants were picked and patched on LB agar plates containing ampicillin (100 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 l of screening primers (to give a final concentration of 100 picomoles) and 0.5 μl of Taq DNA polymerase. pEAK12d clones were screened using the primers pEAK12 FP and INSP181 MAT RP and pDEST12.2 clones were screened using the primers 21M13FP and INSP181 MAT RP.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from a 5 ml culture using QIAprep Spin Miniprep kit (Qiagen).

Plasmid DNA (150-200 ng) in the pEAK12d vector was subjected to DNA sequencing with the sequencing primers pEAK12 FP and pEAK12 RP as described above. Plasmid DNA (150-200 ng) in the pDEST12.2 vector was subjected to DNA sequencing with the sequencing primers 21M13 FP and M13Rev RP as described above.

Sequence confirmed clones were designated as pEAK12d_INSP181SV1-6HIS and pDEST12.2_INSP181SV1-6HIS.

Maxi-prep DNA was prepared from a 500 ml culture of the sequence verified clone pEAK12d_INSP181-6HIS using a Qiagen Maxi prep kit according to the manufacturer's instructions. Plasmid DNA was resuspended at a concentration of 1 μg/μl in TE buffer and stored at −20° C.

Endotoxin-free maxi-prep DNA was prepared from a 500 ml culture of the sequence verified clone pDEST12.2_INSP181-6HIS using the EndoFree Plasmid Mega kit (Qiagen) according to the manufacturer's instructions. Purified plasmid DNA was resuspended in endotoxin free TE buffer at a final concentration of at least 3 μg/μl and stored at −20° C.

### Example 5

### Assays Suitable for Exploration of the Biological Relevance of INSP181 Function

It is believed that the moieties of the invention will be particularly useful for the treatment or diagnosis of disorders/diseases of the reproductive system and autoimmune diseases/disorders. It is believed that the following assays will be useful to test for moieties that have useful biological effects. Note that although some of the following assays refer to the test compound as being a protein/polypeptide, a person skilled in the art will readily be able to adapt the following assays so that the other moieties of the invention may also be used as the “test compound”.

### A Reproductive Health Assays

### JEG-3 Implantation Assay:

In this assay, a 2-chamber system is used where fluorescently labeled JEG-3 cells invade through a Matrigel-coated porous membrane from an upper chamber into a lower chamber when Ishikawa cells or Ishikawa-conditioned medium are placed into the lower chamber. The cells that migrate are quantified in a plate reader. The goal is to identify proteins that increase invasion of JEG-3 cells for use in aiding implantation in vivo.

### Osteopontin Bead Assay (Ishikawa Cells)

In this assay, osteopontin-coated fluorescent beads represent the blastocyst, and the Ishikawa cells are primed to accept them for binding by treating them with estradiol. The goal is to identify proteins that increase the ability of the Ishikawa cells to bind the osteopontin-beads as an aid to increase receptivity of the uterine endometrium at the time of implantation.

### HuF6 Assay:

In this assay the goal is to identify proteins that increase production of PGE2 (a marker for decidualization) by the HuF6 cells as a way of enhancing decidualization during early pregnancy.

### Endometriosis Assay:

Peritoneal TNFa plays a role in endometriosis by inducing the sloughed endometrial cells from the uterus to adhere to and proliferate on peritoneal mesothelial cells. In this assay, BEND cells are treated with TNFa, which increases their ability to bind fibronectin-coated fluorescent beads as an assay for adherence during endometriosis. The goal is to identify proteins that decrease or inhibit the ability of TNFa to stimulate bead-binding capacity of the cells.

Cyclic AMP Assay Using JC-410 Porcine Granulose Cells Stably Transfected with hLHR.

In Polycystic Ovary Syndrome, LH from the pituitary is relatively high, and induces androgen output from the ovarian thecal cells. In this assay, we are looking for an inhibitor of LH signaling which could be used to decrease the action of LH at the ovary during PCOS. The JC-410 porcine granulosa cell line was stably transfected with the human LH receptor. Treatment with LH results in cAMP production.

Cyclic AMP Assay Using JC-410 Porcine Granulose Cells Stably Transfected with hFSHR.

The JC-410 porcine granulosa cell line was stably transfected with the human FSHR. Treatment with FSH stimulates cAMP production, which is measured in this assay. The goal is to identify proteins that enhance FSH action in the granulosa cells.

### LbetaT2 (Mouse) Pituitary Cells Assay.

The LbT2 is an immortalized murine pituitary gonadotroph cell line. Stimulation with Activin alone or with GnRH+Activin results in secretion of FSH. The cells can either be treated with GnRH+Bioscreen proteins to find proteins that act in concert with GnRH to stimulate FSH production, or they can be treated with Bioscreen proteins alone to find a protein that can stimulate FSH secretion like activin alone.

### Cumulus Expansion Assay

Using murine cumulus-oocyte complexes an assay can be developed to identify moieties which promote expansion.

### RWPE Proliferation Assay

Benign prostatic hyperplasia is characterized by growth of prostatic epithelium and stroma that is not balanced by apoptosis, resulting in enlargement of the organ. RWPE is a regular human prostatic epithelial cell line that was immortalized with the HPV-18, and is used in place of primary human prostatic epithelial cells, which are not always available.

### HT-1080 Fibrosarcoma Invasion Assay

Fluorescently-labeled HT-1080 human fibrosarcoma cells are cultured in the upper chamber of a 2-chamber system, and can be stimulated to invade through the porous Matrigel-coated membrane into the bottom chamber where they are quantified. The goal would be to identify a moiety that would inhibit the invasion.

### Primary Human Uterine Smooth Muscle Assay

One of the hallmarks of uterine fibroid disease is collagen deposition by the uterine smooth muscle cells that have become leiomyomas. Primary human uterine smooth muscle cells are stimulated to produce collagen by treatment with TGFb, which is blocked with Rebif. The goal is to discover proteins that inhibit this fibrotic phenotype.

### Human Leiomyoma Cells Proliferation Assay

Human leiomyoma cells may be used as a model for uterine fibroid disease in a proliferation assay. The cells grow very slowly but may be stimulated with estradiol and growth factors. The goal is to identify proteins that inhibit estradiol-dependent growth of leiomyoma cells.

### U937 Migration Assay.

Endometriotic lesions secrete cytokines that recruit immune cells to the peritoneal cavity which then mediate inflammatory symptoms that are common to endometriosis. RANTES has been shown to be produced by endometriotic stromal cells and is present in the peritoneal fluid. In this assay, U937, a monocytic cell line used as a model for activated macrophages, can be induced by treating the lower level of a 2-chamber culture system to migrate from the upper chamber. If the cells are pre-loaded with fluorescent dye, they can be quantified in the lower chamber. The goal will be to identify proteins that inhibit the migration of the U937 cells.

### JEG3 Human Trophoblast Assay

The trophoblast of the blastocyst produces HLA-G, a class I HLA molecule that is believed to be important in preventing immunological rejection of the embryo by the mother. During pre-eclampsia, HLA-G levels are low or non-existent. The JEG-3 human trophoblast cell line produces HLA-G and may be utilised to identify moieties that can increase HLA-G production.

### Primary Rat Ovarian Dispersate Assay

The amount of estradiol production from cultures of cells from whole ovaries taken from immature rats or other rodents may be measured after treatment with FSH and/or LH. The goal will be to identify proteins that enhance gonadotropin-stimulated steroidogenesis, or proteins that work alone to increase steroidogenesis by these cultures.

### Mouse IVF Assay

In this assay, sperm function, measured by ability to fertilize oocytes, will be assayed with the goal of finding proteins that stimulate fertilizing potential of sperm. Such an assay may be run with, for example, mouse sperm and oocytes.

### Primary Human Prostate Stromal Cells Proliferation Assay

An assay for the epithelial component of BPH has already been developed (see RWPE above). This assay uses primary human prostate stromal cells as a model for proliferation of these cells during BPH. The goal will be to identify proteins that inhibit proliferation of these cells.

### Primary Human Uterine Smooth Muscle Proliferation Assay

Proteins and other moieties may be tested to thereby identify moieties capable of inhibiting the proliferation of primary human uterine smooth muscle cells. Proliferation of uterine smooth muscle cells is a precursor for development of tumours in uterine fibroid disease.

B Autoimmune assays

| | | | Biology | Targeted |
|---|---|---|---|---|
| | Cells and stimuli | Read-out | rationale | diseases |
| T lymphocytes | Fas-Ligand-induced | Release of | To regulate T | Autoimmune |
| | Jurkat T cell death. | LDH. | cell death | diseases |
| | Human PBMC | Proliferation | To modulate | Autoimmune |
| | stimulated with the | | T cell | diseases |
| | superantigen, TSST. | | proliferation | |
| | | Cytokine | To modulate | Autoimmune |
| | | secretion. | T cell | diseases |
| | | | cytokine | |
| | | | secretion | |
| T lymphocytes and | Human and Mouse | Proliferation | To modulate | Autoimmune |
| Antigen presenting | MLR | | T cell | diseases |
| cells | | | proliferation | |
| | | Cytokine | To modulate | Autoimmune |
| | | secretion. | T cell | diseases |
| | | | cytokine | |
| | | | secretion | |
| | Human PBMC | Cytokine | To modulate | Autoimmune |
| | stimulated with either | secretion. | T cell | diseases |
| | ConA or PHA. | | cytokine | |
| | | | secretion | |
| Monocytes | Human PBMC | Cytokine | To modulate | Autoimmune |
| macrophages and | stimulated with LPS | secretion. | macrophage | diseases |
| granulocytes | | | and | |
| | | | granulocyte | |
| | | | cytokine | |
| | | | secretion | |
| Monocytes | RANTES-induced | Calcium flux by | To induce | Autoimmune |
| | calcium flux in THP-1. | FlipR | monocyte | diseases |
| | | | activation | |
| Neutrophils | Human neutrophils | Cytoskeleton | To modulate | Autoimmune |
| | stimulated with IL-8 | reorganization | neutrophil | diseases |
| | | | migration | |
| B lymphocytes | Human B cell | Survival | To modulate | Autoimmune |
| | stimulated with goat | | B cell | diseases |
| | anti-human IgM | | survival | |
| | antibody and rhIL-4 | | | |
| | Human B cell | Proliferation | To modulate | Autoimmune |
| | stimulated plus goat | | B cell | diseases |
| | anti-human IgM | | costimulation | |
| | antibody, rhIL-4 and | | | |
| | soluble rhBAFF | | | |
| Microglia cells | M-CSF activated | Proliferation | To modulate | MS |
| | microglia cell line | | microglia cell | |
| | | | activation | |

### Assays Targeting T Lymphocyte Responses

Fas-Ligand-induced T cell death. This assay will reveal new modulators of receptor mediated cell death. In this assay, T cell apoptosis is induced by stimulating Jurkat cells with recombinant 6 Histidine-tagged Fas Ligand combined with a monoclonal anti 6-his antibody. Death is quantified by release of LDH, a cytoplasmic enzyme released in the culture medium when cells are dying. T cells have been shown to be pathogenic in many autoimmune diseases, being able to control antigen-specific T cell death is a therapeutic strategy.
Human-MLR: proliferation and cytokine secretion. This cell-based assay measures the effects of novel proteins on lymphocyte proliferation and cytokine secretion or inhibition upon stimulation by PBMC from another donor (alloreactivity).
Human PBMC stimulated with the superantigen, TSST.

In this cellular assay, T lymphocyte activation may be specifically targeted via the TCR but with different requirements than the T cell response to classical antigens, in particular in respect to co-stimulatory molecules.
Human PBMC stimulated with either ConA or PHA. These cell-based assays measure the effects of novel proteins on cytokine secretion induced by two different stimuli acting on different cells as measured by a cytokine bead array (CBA) assay (IL-2, IFN-γ, TNF-α, IL-5, IL-4 and IL-10).

### Assays Targeting Monocyte/Macrophages and Granulocyte Responses

Human PBMC stimulated with LPS. This cell-based assay measures the effects of novel proteins on cytokine secretion (IFN-γ, TNF-α) induced by LPS acting on monocytes/macrophages and granulocytes.

### Assays Targeting Neutrophil Responses

The tissue infiltration of neutrophils depends on a reorganisation of cytoskeleton elements associated with specific changes in cell morphology of these cells. This cell-based assay measures the effect of novel proteins on cytoskeleton reorganization of human neutrophils.

### Assays Targeting B Lymphocyte Responses

B cell proliferation. This cell-based assay measures the effect of novel proteins on B cell survival.
B cell co-stimulation. This cell-based assay measures the effect of novel proteins on B cell co-stimulation.

### Assays Targeting Monocytes and Microglial Responses

THP-1 calcium flux. The Ca⁺-flux in THP1-cell assay measures the effects of novel proteins on their ability to trigger an intracellular calcium release (a generic second messenger event) from the endoplasmic reticulum.
Microglia cell proliferation

During proliferation of microglial progenitors, a number of colony-stimulating factors, including some cytokines, are known to play key roles. Among them, M-CSF is crucial for the final step of maturation of macrophages/microglia and is not replaceable by any other factor. The evaluation of this biological response may represent a way to influence the microglial activity and therefore an opportunity to identify molecules with therapeutic potential from MS. Person skilled in the art will be able to develop a cell-based assay which can measure the proliferative response of a microglia cell line to M-CSF.

Other assays which may be useful include a cytokine expression modulation assay. Briefly, the effects of the test protein (or other test moiety) on cytokine secretion induced by Concanavalin A acting on different human peripheral blood mononuclear cells (hPBMC) cells as measured by a cytokine bead array (CBA) assay for IL-2, IFN-γ, TNF-α, IL-5, IL-4 and IL-10 are measured. Using such an assay, the “best inhibited” cytokine can be determined and the diseases correlated with such cytokine can be found in the literature.

### Example 5

### Effect of INSP181 on Cytokine Secretion by Con A Stimulated PBMCs

### 5.1 Summary

INSP181 was tested for its effect on cytokine secretion by Human Peripheral Blood Mononuclear Cells (PBMC) stimulated with the mitogen, concanavalin A (ConA). INSP181-6HIS stimulates IL-10, IL-4 and IL-5 secretion from ConA-stimulated human PBMC when tested at a 1/10 dilution (46.2 ug in the assay). No effect was seen on the levels of IFN-γ, TNF-α, or IL-2.

### 5.2 Materials and Reagents

Buffy coat
DMEM GIBCO Ref: 21331-020
Human serum type AB SIGMA Ref: H1513
L-Glutamine GIBCO Ref: 250 030-020
Penicillin-Streptomycin GIBCO Ref: 150 070-063
Ficoll PHARMACIA ref: 17-1440-03
96 well microtiter plate for cell culture COSTAR Ref: 3596
Concanavalin A SIGMA Ref: C0412
Dexamethasone water soluble SIGMA Ref: D2915
Human Th1/Th2 Cytokine CBA Kit Becton-Dickinson Ref: 550749
PBS GIBCO Ref: 14190-094
FALCON 50 ml sterile Becton-Dickinson Ref: 2070
Glycerol MERCK Ref: 1-04092-2500
96 well microtiter plate conical bottom NUNC Ref: 249570

### 5.3 Method

5.3.1 Purification of Human PBMC from a Buffy Coat

Dilute the buffy coat 1 to 2 with DMEM.

Slowly add 25 ml of diluted blood onto a 15 ml layer of Ficoll in a 50 ml Falcon tube.

Centrifuge the tubes (2000 rpm, 20 min, at RT without brake).

Collect the interphase (ring) and wash the cells with 25 ml of DMEM followed by a centrifuge step (1200 rpm, 5 min). Repeat 3 times. A buffy coat would give approximately 600×10⁶ total cells.

### 5.3.2 Activity Test

Add 80 μl of 1.25×10⁶ cells/ml, diluted in DMEM+2.5% Human Serum+1% L-Glutamine+1% Penicillin-Streptomycin, to a 96 well microtiter plate.

Add 10 μl per well (one condition per well): AS902285/1 in PBS+20% Glycerol

Add 10 μl per well: ConA 50 μg/ml (the final concentration of ConA is 5 μg/ml)

After 48 h, cell supernatants are collected and human cytokines measured by Human Th1/Th2 Cytokine CBA Kit Becton-Dickinson.

### 5.3.3 CBA Analysis

Human Th1/Th2 Capture Beads mixture is prepared following the supplier instructions (CBA Kit Becton-Dickinson Ref: 550749), briefly:
Determine the number of assay tubes that are required for the experiment.
Vigorously vortex each capture bead suspension for a few seconds before mixing.
Add a 10 μl aliquot of each capture bead, for each assay to be analysed, into a single tube labelled “mixed capture beads”.
Vortex the Bead mixture thoroughly.

### Preparation of Test Samples

Dilute supernatants 1:5 using the Assay Diluent (20 μl of supernatants+60 μl of Assay Diluent).
Mix sample dilution before transferring samples into a 96 wells microtiter plate conical bottom. (Nunc)

### Human Th1/Th2 Cytokine CBA Assay Procedure

Add 50 μl of the diluted supernatants into a 96 wells microtiter plate conical bottom (Nunc).
Add 50 μl of the mixed capture beads.
Add 50 μl of the Human Th1/Th2 PE Detection Reagent.
Incubate the plate for 3 hours at RT and protect from direct exposure to light.
Centrifuge at 1500 rpm for 5 minutes.
Carefully discard the supernatant.
Add 200 μl of wash buffer to each well and centrifuge at 1500 rpm for 5 minutes.
Carefully discard the supernatant.
Add 200 μl of Wash Buffer to each well and centrifuge at 1500 rpm for 5 minutes.
Carefully discard the supernatant.
Add 130 μl of wash buffer to each well to resuspend the bead pellet.
Analyse samples on a flow cytometer.
The data are analysed using the CBA Application Software, Activity Base and Microsoft Excel software.
The results are given in percentage of cytokine secretion compare to the level of cytokine achieved by ConA stimulation (100%) versus non stimulated cells (0%).

### 5.4. Results

In one experiment, INSP181-6HIS stimulated secretion of IL-10, (196%), and the Th2 cytokines IL-4 (257%) and IL-5 (165%) from Con A stimulated PBMCs, but had no effect on IFN-γ, TNF-α or IL-2 secretion measured using the CBA assay (Table 3). Thus, the present invention is based on the finding that polypeptides of the present invention upregulate Th2 cytokines, more specifically interleukin-10 (IL-10), interleukin-4 (IL-4) and interleukin-5 (IL-5). In addition, this upregulation is specific for Th2 cytokines as Th1 cytokines' levels (i.e. IFN-γ, TNF-α or IL-2) remain unchanged. This specific profile of cytokine expression leads to the potential therapeutic use of the polypeptides of the present invention in Th1 diseases, antagonists thereof being useful in Th2 diseases.

TABLE 3
Effect of INSP181-6HIS on cytokine secretion by Con A stimulated human
PBMCs

| Assay | Protocol | Plate/well | Rep | % Stim | StdDev | Conc |
|---|---|---|---|---|---|---|
| CELL-CBA | CON-HPBL-IFN-10-02 | MP-9089/G05 | 1 | 96.00% | N/A | .1 dilution |
| CELL-CBA | CON-HPBL-IL10-10-02 | MP-9089/G05 | 1 | 196.00% | N/A | .1 dilution |
| CELL-CBA | CON-HPBL-IL2-10-02 | MP-9089/G05 | 1 | 107.00% | N/A | .1 dilution |
| CELL-CBA | CON-HPBL-IL4-10-02 | MP-9089/G05 | 1 | 257.00% | N/A | .1 dilution |
| CELL-CBA | CON-HPBL-IL5-10-02 | MP-9089/G05 | 1 | 165.00% | N/A | .1 dilution |
| CELL-CBA | CON-HPBL-TNF-10-02 | MP-9089/G05 | 1 | 138.00% | N/A | .1 dilution |

### Example 6

### 6.1 Analysis of INSP181 Gene Expression Levels by Real Time PCR (Taqman)

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, and dTTP, and DEPC-treated water in a volume of 10 μl. The mixture was incubated at 65° C. for 5 min then chilled on ice for 1 min. The following 10 μl cDNA synthesis mix was prepared in a separate tube: 2 μl 10×RT buffer, 4 μl 25 mM MgCl₂, 2 μl 0.1M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700). PCR primers for human INSP181 and the housekeeping control gene glyceraldehyde 3-phosphate dehydrogenase (GAPDH) were designed using the Primer Express software (PE Biosystems). The forward primer was designed in exon 1. The reverse primer was designed in the exon 2. This primer will not distinguish INSP181 from INSP181SV.

The sequences of the primers are shown in Table 4. The specificity and the optimal primer concentration to use for the TaqMan analysis were determined by testing the INSP181 primers on a series of dilutions of plasmid pEAK12d-INSP181-6HIS and pEAK12d-INSP181SV-6HIS. Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the INSP181 gene in each cDNA sample was expressed as a difference in Ct value, i.e. Delta (δ) Ct=Ct (GAPDH)−Ct (INSP181). Results for each sample were then expressed as a fold difference in the number of cycles required for detectable INSP181 gene expression relative to that for GAPDH, according to the formula Fold Difference=2^{(-δCt)}. Finally, the expression level of the INSP181 gene in each cDNA sample was shown relative to the GAPDH gene expression level, where GAPDH expression level=100%, by dividing 100 by the Fold Difference for INSP181.

### 6.2 Results

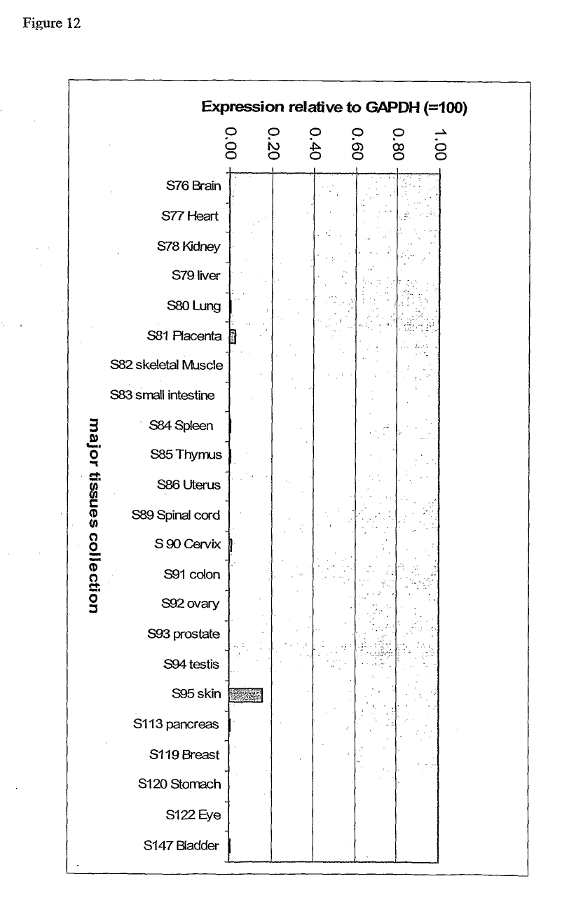

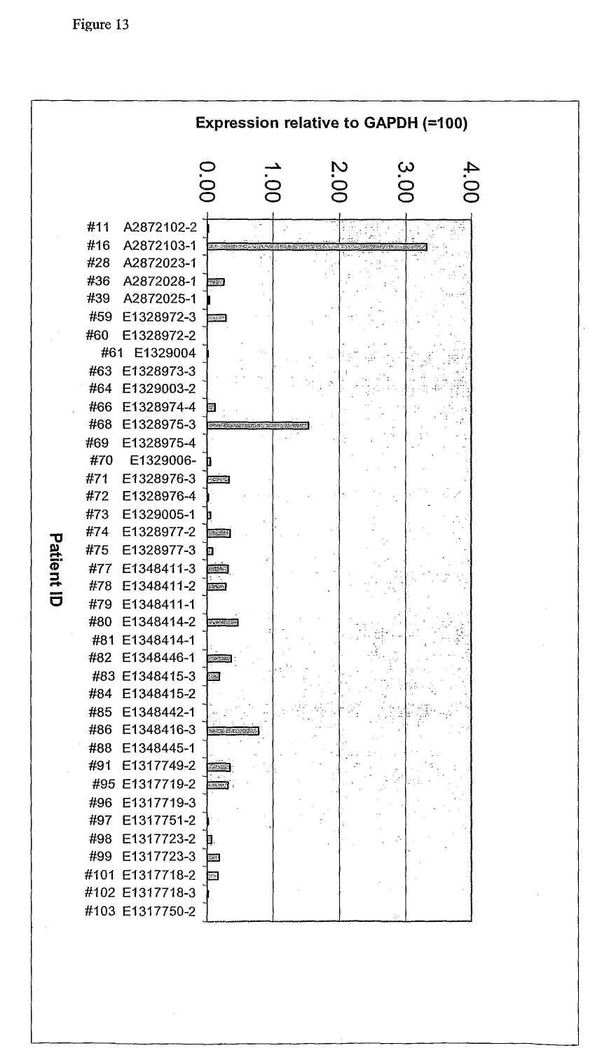

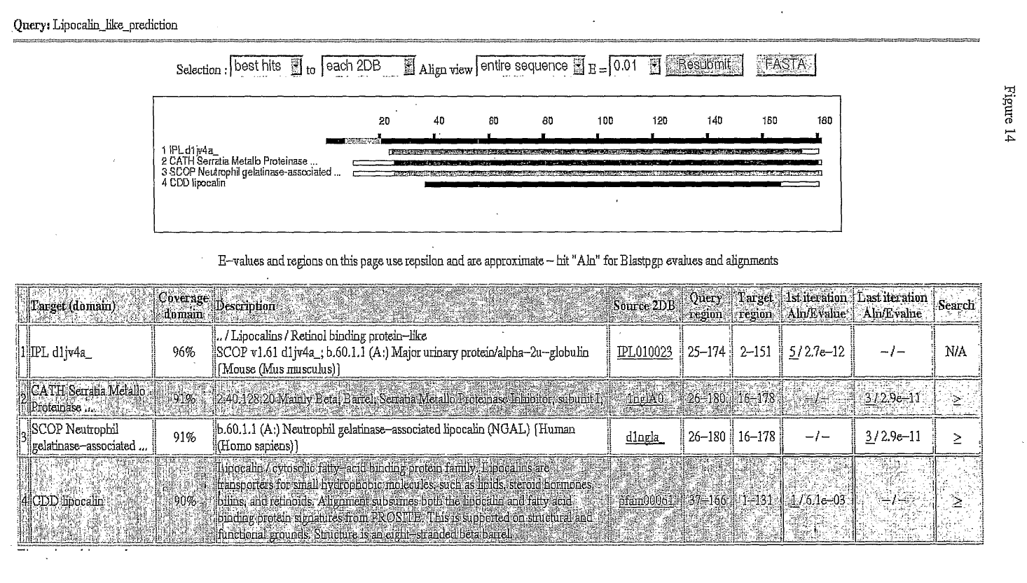

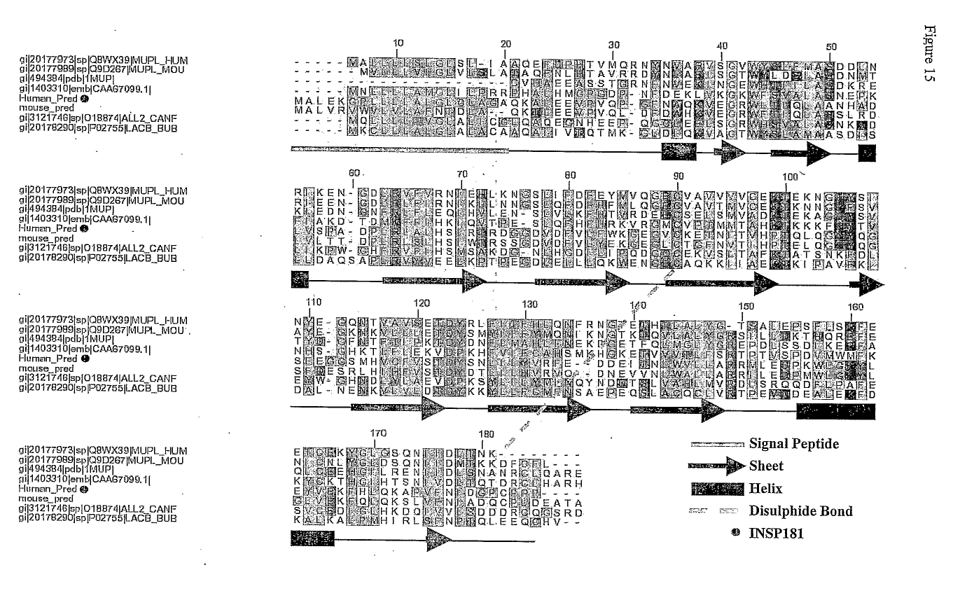

INSP181 primers were tested on a panel of approximately 100 normal and diseased human tissue samples, primary cells and cell lines in addition to 44 inflammatory bowel disease colon and ileum biopsies and 39 psoriasis biopsies from an IL18BP clinical trial. Results are shown in tables 5 and 6 and represented graphically in FIGS. 12 and 13. INSP181 expression was surprisingly only detected at low level in skin (0.16% of GAPDH) (Table 5, FIG. 12) and in skin biopsies from psoriasis patients (19/39 samples positive) (table 6, FIG. 13). A second primer pair specific for exons 4/6 (forward primer in exon 4 and reverse primer in exon 6) confirmed the skin specificity.

Expression results show unexpected restricted expression of INSP81 in skin biopsy samples and psoriasis skin biopsies.

This specific pattern of expression leads to the conclusion of the involvement of INSP181 in skin diseases. Preferably, the skin disease is a Th1 or Th2 skin disease. Preferably, the Th1 disease is psoriasis.

These surprising properties characterizing the polynucleotides or the corresponding polypeptides of the present invention make them particularly suitable for the preparation of a drug or pharmaceutical composition. The polynucleotides or the corresponding polypeptides of the present invention therefore display the unexpected finding of a restricted expression in specific tissues.

TABLE 4

TaqMan PCR primer sequences

| | |
|---|---|
| Primer | Sequence (5′-3′) |

| | |
|---|---|
| h-INSP181-57F4 | TGCCCAGAAGGCTCTGGAA |
| h-INSP181-199R4 | GAGTGGAGAGCGAGCCTCAG |
| hGAPDH-F | GCACCCATGGCAAATTCC |
| hGAPDH-R | GATGGGATTTCCATTGATGACA |
| Intron-hGAPDH-F | CCTAGTCCCAGGGCTTTGATT |
| Intron-hGAPDH-R | GTGTGCTCCCACTCCTGATTT |

TABLE 5
Expression of INSP181 in major human tissues as measured by RT-PCR
(TaqMan).

| Normal human | | Ct | delta | | Relative to |
|---|---|---|---|---|---|
| tissues | Ct hGAPDH | hINSP181 | ct | Fold difference | GAPDH (=100) |
| S76 Brain | 19.44 | 35.87 | −16.43 | 88307.33 | 0.00 |
| S77 Heart | 20.16 | 36.81 | −16.65 | 102847.63 | 0.00 |
| S78 Kidney | 19.41 | 34.71 | −15.30 | 40248.46 | 0.00 |
| S79 liver | 20.93 | 37.96 | −17.03 | 133696.30 | 0.00 |
| S80 Lung | 21.65 | 36.46 | −14.81 | 28811.12 | 0.00 |
| S81 Placenta | 21.71 | 33.42 | −11.71 | 3345.11 | 0.03 |
| S82 skeletal | 15.59 | 37.22 | −21.63 | 3251044.79 | 0.00 |
| Muscle | | | | | |
| S83 small | 18.27 | 35.85 | −17.59 | 196700.49 | 0.00 |
| intestine | | | | | |
| S84 Spleen | 21.63 | 35.17 | −13.54 | 11885.82 | 0.01 |
| S85 Thymus | 19.36 | 33.64 | −14.28 | 19884.13 | 0.01 |
| S86 Uterus | 19.76 | 38.67 | −18.91 | 493684.90 | 0.00 |
| S89 Spinal | 19.75 | 35.61 | −15.87 | 59718.96 | 0.00 |
| cord | | | | | |
| S 90 Cervix | 22.17 | 34.98 | −12.81 | 7179.00 | 0.01 |
| S91 colon | 19.67 | 36.65 | −16.99 | 129964.13 | 0.00 |
| S92 ovary | 20.84 | 37.72 | −16.88 | 120767.86 | 0.00 |
| S93 prostate | 19.80 | 35.88 | −16.08 | 69164.90 | 0.00 |
| S94 testis | 20.05 | 35.80 | −15.75 | 55167.63 | 0.00 |
| S95 skin | 23.39 | 32.72 | −9.33 | 641.93 | 0.16 |
| S113 | 21.12 | 36.18 | −15.06 | 34140.49 | 0.00 |
| pancreas | | | | | |
| S119 Breast | 20.28 | 39.91 | −19.62 | 806755.23 | 0.00 |
| S120 Stomach | 21.80 | 38.54 | −16.74 | 109334.52 | 0.00 |
| S122 Eye | 21.16 | 37.70 | −16.54 | 95534.29 | 0.00 |
| S147 Bladder | 19.21 | 33.69 | −14.48 | 22777.41 | 0.00 |

TABLE 6
Expression of INSP181 in diseased skin biopsies from IL18BP clinical trial as
measured by RT-PCR (TaqMan)

| | | Ct | Ct | | Fold | Relative |
|---|---|---|---|---|---|---|
| Psoriasis | | hGAPDH | hINSP181 | delta ct | difference | to GAPDH (=100) |
| #11 | A2872102-2 | 20.90 | 34.36 | −13.46 | 11306.74 | 0.01 |
| #16 | A2872103-1 | 24.39 | 29.31 | −4.92 | 30.19 | 3.31 |
| #28 | A2872023-1 | 22.85 | 38.42 | −15.57 | 48690.17 | 0.00 |
| #36 | A2872028-1 | 25.37 | 34.04 | −8.67 | 406.67 | 0.25 |
| #39 | A2872025-1 | 22.41 | 34.00 | −11.59 | 3089.02 | 0.03 |
| #59 | E1328972-3 | 23.46 | 31.89 | −8.44 | 346.19 | 0.29 |
| #60 | E1328972-2 | 21.22 | 37.35 | −16.12 | 71255.76 | 0.00 |
| #61 | E1329004 | 25.11 | 38.12 | −13.00 | 8203.29 | 0.01 |
| #63 | E1328973-3 | 23.15 | 40.00 | −16.85 | 118472.89 | 0.00 |
| #64 | E1329003-2 | 21.05 | 36.32 | −15.27 | 39647.25 | 0.00 |
| #66 | E1328974-4 | 22.28 | 32.06 | −9.78 | 881.12 | 0.11 |
| #68 | E1328975-3 | 22.80 | 28.83 | −6.03 | 65.14 | 1.54 |
| #69 | E1328975-4 | 23.75 | 39.20 | −15.45 | 44792.00 | 0.00 |
| #70 | E1329006-1 | 25.60 | 36.62 | −11.02 | 2075.71 | 0.05 |
| #71 | E1328976-3 | 24.41 | 32.68 | −8.27 | 308.23 | 0.32 |
| #72 | E1328976-4 | 23.01 | 35.38 | −12.37 | 5282.35 | 0.02 |
| #73 | E1329005-1 | 23.67 | 34.77 | −11.10 | 2193.42 | 0.05 |
| #74 | E1328977-2 | 24.00 | 32.14 | −8.14 | 282.24 | 0.35 |
| #75 | E1328977-3 | 21.88 | 32.04 | −10.16 | 1146.47 | 0.09 |
| #77 | E1348411-3 | 23.19 | 31.49 | −8.30 | 315.93 | 0.32 |
| #78 | E1348411-2 | 23.19 | 31.63 | −8.44 | 346.92 | 0.29 |
| #79 | E1348411-1 | 18.93 | 35.83 | −16.91 | 122965.58 | 0.00 |
| #80 | E1348414-2 | 21.24 | 29.00 | −7.76 | 216.69 | 0.46 |
| #81 | E1348414-1 | 21.07 | 40.00 | −18.93 | 500034.81 | 0.00 |
| #82 | E1348446-1 | 20.77 | 28.87 | −8.10 | 274.63 | 0.36 |
| #83 | E1348415-3 | 20.77 | 29.81 | −9.04 | 526.35 | 0.19 |
| #84 | E1348415-2 | 18.54 | 32.15 | −13.60 | 12438.44 | 0.01 |
| #85 | E1348442-1 | 19.84 | 34.46 | −14.61 | 25074.26 | 0.00 |
| #86 | E1348416-3 | 21.58 | 28.59 | −7.02 | 129.48 | 0.77 |
| #88 | E1348445-1 | 21.55 | 39.17 | −17.61 | 200512.76 | 0.00 |
| #91 | E1317749-2 | 24.51 | 32.70 | −8.20 | 293.22 | 0.34 |
| #95 | E1317719-2 | 25.37 | 33.66 | −8.29 | 312.71 | 0.32 |
| #96 | E1317719-3 | 23.52 | 40.00 | −16.48 | 91584.99 | 0.00 |
| #97 | E1317751-2 | 22.80 | 35.40 | −12.60 | 6201.23 | 0.02 |
| #98 | E1317723-2 | 25.21 | 35.90 | −10.70 | 1657.76 | 0.06 |
| #99 | E1317723-3 | 23.86 | 32.91 | −9.06 | 532.89 | 0.19 |
| #101 | E1317718-2 | 20.80 | 30.04 | −9.24 | 606.53 | 0.16 |
| #102 | E1317718-3 | 22.86 | 36.13 | −13.27 | 9887.33 | 0.01 |
| #103 | E1317750-2 | 20.94 | 35.46 | −14.52 | 23478.87 | 0.00 |

### Example 7

### Microarray Studies

Custom microarrays have been manufactured using Agilent Technologies' (Agilent Technologies Inc, Palo Alto, Calif.) non-contact in situ synthesis process of printing 60-mer length oligonucleotide probes, base-by-base, from digital sequence files. This is achieved with an inkjet process which delivers extremely small, accurate volumes (picoliters) of the chemicals to be spotted. Standard phosphoramidite chemistry used in the reactions allows for very high coupling efficiencies to be maintained at each step in the synthesis of the full-length oligonucleotide. Precise quantities are reproducibly deposited “on the fly.” This engineering feat is achieved without stopping to make contact with the slide surface and without introducing surface-contact feature anomalies, resulting in consistent spot uniformity and traceability. (Hughes et al. (2001) Nat. Biotech. April; 19(4): 342-7. Expression profiling using microarrays fabricated by an ink-jet oligonucleotide synthesizer).

### Probe Synthesis

Methodologies were carried out according to Agilent instructions. Essentially, cDNA synthesis and subsequent T7 polymerase amplification of Cyanine 3(5)-CTP labeled cRNA probe was carried out using Agilent's low RNA input fluorescent linear amplification kit from a template of 5 μg of total RNA according to the kit protocol (version 2 Aug. 2003, Agilent, Palo Alto, Calif.). cRNA is then fragmented using Agilent's In Situ hybridization kit-plus and hybridized both according to Agilent's protocol (Agilent 60-mer oligo microarray processing protocol version 4.1 April 2004, Agilent, Palo Alto, Calif.).

### Microarray Chip Design

10,536 probes are on the array
5557 of the probes designed specifically to detect secreted sequences of primary interest
1000 probes designed as negative controls
500 probes designed as positive controls
Remainder of the probes were designed to public domain sequences which are known to be either secreted soluble extracellular proteins or membrane bound proteins with an extracellular domain in contact with the extracellular milieu.

### Studies Specific for INSP181

INSP181 is formed from separate component exons. We intend to profile the chips using probe synthesized from 10 normal tissues, bone marrow, brain, lung, ovary, PBMCs, placenta, prostate, spleen and testis. Expression reports are obtainable on an exon by exon basis.

Averaging is performed for the data, using the One-step Tukey Bi-Weight Algorithm (Data Analysis and Regression: A Second Course in Statistics”, Mosteller and Tukey, Addison-Wesley, 1977, pp. 203-209; see also Affymetrix MAS5.0 algorithm). The purpose of this is to define a robust estimate of the average value of a dataset. In this case our datasets will comprise multiple probe expression values for a single exon.

This custom array is useful for a number of reasons. First, it allows the existence and sequence of the transcript to be confirmed. Second, the tissue distribution of the INSP181 polypeptide sequence can be evaluated and thus the role of this polypeptide in disease can be clarified. The array can also be used as a diagnostic tool, to diagnose disease incidence in patients with disease conditions with which this polypeptide is correlated. The use of exon-specific probes allows any variance in expression of splice variants of this polypeptide sequence to be evaluated, in general, in specific tissues and in specific disease states.

We claim:

1-48. (canceled)

49: A composition of matter comprising:
a) an isolated polypeptide selected from:
1) an amino acid sequence comprising that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
2) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
3) a fragment of 1) or 2), wherein the fragment is a lipocalin or that has an antigenic determinant in common with the polypeptide according to 1) or 2); or
4) a functional equivalent of 1), 2), or 3); or
5) the fragment of 3), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
6) the fragment of 3), wherein the fragment is a lipocalin and consists of amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
7) the functional equivalent of 4), wherein the functional equivalent is a lipocalin and is homologous to the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
8) the fragment or functional equivalent of 3), 4), 5), or 6), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
9) the fragment or functional equivalent of 3), 4), 5), or 6), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
10) the fragment or functional equivalent of 3), 4), 5), or 6), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
11) the fragment or functional equivalent of 3), 4), 5), or 6), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
12) the fragment or functional equivalent of 3), 4), 5), or 6), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
13) the polypeptide of any of 1) to 7), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
14) the fragment of any of 3), 8), 9), 10), 11), or 12), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
15) the polypeptide of any of 1) to 14), wherein the polypeptide contains amino acid substitution N92T, G114S, or both; or
16) the fragment of 14), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
17) a fusion polypeptide comprising the polypeptide according to any of 1) to 16); or
18) the of any of 1) to 17), further comprising a histidine tag; or
19) the polypeptide of 18), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72; or
b) a purified nucleic acid molecule:
1) comprising a nucleic acid sequence encoding a polypeptide of any one of a1) to a19); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) that hybridizes under high stringency conditions with a nucleic acid molecule of any of b1) to b3); or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to a19); or
2) that binds specifically to the polypeptide of any of a1) to a19) and modulates the activity of the polypeptide; or
3) which is an antibody that binds specifically to the polypeptide of any of a1) to a19); or
4) which is an antibody that binds specifically to the polypeptide of any of a1) to a19), and modulates the activity of the polypeptide; or
f) a compound:
1) that increases the level of expression or activity of a polypeptide according to any of a1) to a19); or
2) that decreases the level of expression or activity of a polypeptide according to any of a1) to a19); or
g) a compound that binds to a polypeptide according to any of a1) to a19) without inducing any of the biological effects of the polypeptide; or
h) a compound that binds to a polypeptide according to any of a1) to a19) without inducing any of the biological effects of the polypeptide, wherein the compound is a natural or modified substrate, ligand, enzyme, receptor, or structural or functional mimetic; or
i) a pharmaceutical composition comprising any one of a) to h), and a pharmaceutically acceptable carrier; or
j) a vaccine composition comprising any one of a1) to a19) or b1) to b4); or
k) a kit useful for diagnosing disease, comprising a first container containing a nucleic acid probe that hybridizes under stringent conditions with a nucleic acid molecule of any one of b1) to b4), a second container containing primers useful for amplifying the nucleic acid molecule, and instructions for using the probe and primers for facilitating the diagnosis of disease; or
l) a kit useful for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
m) a kit comprising an array of nucleic acid molecules, at least one of which is a nucleic acid molecule according to any one of b1) to b4); or
n) a kit comprising one or more antibodies that bind to a polypeptide as recited in any one of a1) to a19); and a reagent useful for the detection of a binding reaction between the one or more antibodies and the polypeptide; or
o) a transgenic or knockout non-human animal that has been transformed to express higher, lower, or absent levels of a polypeptide according to any one of a1) to a19).

50: A method of using a composition of matter, comprising obtaining a composition of matter according to claim 49 and using said composition of matter in a method selected from the group consisting of: diagnosing a disease in a patient; treatment of a disease in a patient; monitoring the therapeutic treatment of a disease; identification of a compound that is effective in the treatment and/or diagnosis of a disease; and screening candidate compounds for a compound effective to treat a disease.

51: The method of claim 50, wherein said method of using a composition of matter comprises the method for treatment of a disease, comprising administering to the patient:
a) an isolated polypeptide selected from:
1) an amino acid sequence comprising that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
2) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
3) a fragment of 1) or 2), wherein the fragment is a lipocalin or that has an antigenic determinant in common with the polypeptide according to 1) or 2); or
4) a functional equivalent of 1), 2), or 3); or
5) the fragment of 3), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
6) the fragment of 3), wherein the fragment is a lipocalin and consists of amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
7) the functional equivalent of 4), wherein the functional equivalent is a lipocalin and is homologous to the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
8) the fragment or functional equivalent of 3), 4), 5), or 6), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
9) the fragment or functional equivalent of 3), 4), 5), or 6), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
10) the fragment or functional equivalent of 3), 4), 5), or 6), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
11) the fragment or functional equivalent of 3), 4), 5), or 6), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
12) the fragment or functional equivalent of 3), 4), 5), or 6), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
13) the polypeptide of any of 1) to 7), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
14) the fragment of any of 3), 8), 9), 10), 11), or 12), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
15) the polypeptide of any of 1) to 14), wherein the polypeptide contains amino acid substitution N92T, G114S, or both; or
16) the fragment of 14), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
17) a fusion polypeptide comprising the polypeptide according to any of 1) to 16); or
18) the of any of 1) to 17), further comprising a histidine tag; or
19) the polypeptide of 18), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72; or
b) a purified nucleic acid molecule:
1) comprising a nucleic acid sequence encoding a polypeptide of any one of a1) to a19); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) that hybridizes under high stringency conditions with a nucleic acid molecule of any of b1) to b3); or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to a19); or
2) that binds specifically to the polypeptide of any of a1) to a19) and modulates the activity of the polypeptide; or
3) which is an antibody that binds specifically to the polypeptide of any of a1) to a19); or
4) which is an antibody that binds specifically to the polypeptide of any of a1) to a19), and modulates the activity of the polypeptide; or
f) a compound:
1) that increases the level of expression or activity of a polypeptide according to any of a1) to a19); or
2) that decreases the level of expression or activity of a polypeptide according to any of a1) to a19); or
g) a compound that binds to a polypeptide according to any of a1) to a19) without inducing any of the biological effects of the polypeptide; or
h) a compound that binds to a polypeptide according to any of a1) to a19) without inducing any of the biological effects of the polypeptide, wherein the compound is a natural or modified substrate, ligand, enzyme, receptor, or structural or functional mimetic; or
i) a pharmaceutical composition comprising any one of a) to h), and a pharmaceutically acceptable carrier.

52: The method of claim 51, wherein the disease is selected from among vision disorders (e.g., nightblindness), immune system disorders (e.g., autoimmune disorders), inflammatory disorders, inflammatory bowel disease (IBD), ulcerative colitis (UC), Crohn's disease (CD), proctitis, cell proliferative disorders, cancer (e.g., breast cancer, cutaneous T-cell lymphoma, squamous cell carcinoma and/or basal cell carcinoma), microbial infections (e.g., viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to (e.g., nickel or gold, cutaneous T-cell lymphoma, atopic eczema, acute eczema and/or chronic eczema), reproductive disorders (e.g., infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis, and/or viral infections.

53: The method of claim 51, wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

54: The method of claim 51, wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

55: The method of claim 50, wherein said method of using a composition of matter comprises the method for diagnosing a disease in a patient, comprising assessing the level of expression of a natural gene encoding a polypeptide, or assessing the activity of the polypeptide, in tissue from said patient; and comparing said level of expression or activity to a control level, wherein a level that is different to said control level is indicative of disease, and wherein the polypeptide comprises:
a) the amino acid sequence comprising that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
b) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
c) a fragment of a) or b), wherein the fragment is a lipocalin or that has an antigenic determinant in common with the polypeptide according to a) or b); or
d) a functional equivalent of a), b), or c); or
e) the fragment of c), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
f) the fragment of c), wherein the fragment is a lipocalin and consists of amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
g) the functional equivalent of e), wherein the functional equivalent is a lipocalin and is homologous to the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
h) the fragment or functional equivalent of c), d), e), or f), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
i) the fragment or functional equivalent of c), d), e), or f), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
j) the fragment or functional equivalent of c), d), e), or f), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
k) the fragment or functional equivalent of c), d), e), or f), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
l) the fragment or functional equivalent of c), d), e), or f), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
m) the polypeptide of any of a) to g), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
n) the fragment of any of c), h), i), j), k), or l), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8. SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
o) the polypeptide of any of a) to n), wherein the polypeptide contains amino acid substitution N92T, G114S, or both; or
p) the fragment of n), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
q) a fusion polypeptide comprising the polypeptide according to any of a) to p); or
r) the of any of a) to p), further comprising a histidine tag; or
s) the polypeptide of r), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72.

56: The method of claim 55, which is carried out in vitro.

57: The method of claim 55, comprising:
a) contacting a ligand with a biological sample under conditions suitable for the formation of a ligand-polypeptide complex; and
b) detecting said complex, wherein the ligand binds specifically to the polypeptide of any of a) to s) of claim 55, or wherein the ligand is an antibody that binds specifically to the polypeptide of any of a) to s) of claim 55.

58: The method of claim 55, comprising:
a) contacting a sample of tissue from the patient with a nucleic acid probe under stringent conditions that allow the formation of a hybrid complex between a nucleic acid molecule and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease, wherein the nucleic acid molecule:
1) comprises a nucleic acid sequence encoding a polypeptide of any one of a) to s) of claim 55; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15. SEQ ID NO:17, SEQ ID NO:19, SEQ ID NO:21, SEQ ID NO:23, SEQ ID NO:25, SEQ ID NO:27. SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) hybridizes under high stringency conditions with a nucleic acid molecule of any of c1) to c3).

59: The method of claim 55, comprising:
a) contacting a sample of nucleic acid from tissue of the patient with a nucleic acid primer under stringent conditions that allow the formation of a hybrid complex between a nucleic acid molecule and the primer;
b) contacting a control sample with said primer under the same conditions used in step a);
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease, wherein the nucleic acid molecule:
1) comprises a nucleic acid sequence encoding a polypeptide of any one of a) to s) of claim 55; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) hybridizes under high stringency conditions with a nucleic acid molecule of any of d1) to d3).

60: The method of claim 55, comprising:
a) obtaining a tissue sample from a patient being tested for disease;
b) isolating a nucleic acid molecule from said tissue sample; and
c) diagnosing the patient for disease by detecting the presence of a mutation which is associated with disease in the nucleic acid molecule as an indication of the disease, wherein the nucleic acid molecule:
1) comprises a nucleic acid sequence encoding a polypeptide of any one of a) to s) of claim 55; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29. SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) hybridizes under high stringency conditions with a nucleic acid molecule of any of c1) to c3).

61: The method of claim 60, further comprising amplifying the nucleic acid molecule to form an amplified product and detecting the presence or absence of a mutation in the amplified product.

62: The method of claim 60, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

63: The method of claim 60, wherein said disease is selected from among vision disorders (e.g., nightblindness), immune system disorders (e.g., autoimmune disorders), inflammatory disorders, inflammatory bowel disease (IBD), ulcerative colitis (UC), Crohn's disease (CD), proctitis, cell proliferative disorders, cancer (e.g., breast cancer, cutaneous T-cell lymphoma, squamous cell carcinoma and/or basal cell carcinoma), microbial infections (e.g., viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to e.g., nickel or gold, cutaneous T-cell lymphoma, atopic eczema, acute eczema and/or chronic eczema), reproductive disorders (e.g., infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonepliritis and/or viral infections.

64: The method of claim 50, wherein said method of using a composition of matter comprises the method of monitoring the therapeutic treatment of a disease, comprising monitoring over a period of time the level of expression or activity of a polypeptide, or the level of expression of a nucleic acid molecule, in tissue from said patient, wherein altering said level of expression or activity over the period of time towards a control level is indicative of regression of said disease, wherein
a) the polypeptide comprises:
1) an amino acid sequence comprising that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
2) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
3) a fragment of 1) or 2), wherein the fragment is a lipocalin or that has an antigenic determinant in common with the polypeptide according to 1) or 2); or
4) a functional equivalent of 1), 2), or 3); or
5) the fragment of 3), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
6) the fragment of 3), wherein the fragment is a lipocalin and consists of amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
7) the functional equivalent of 4), wherein the functional equivalent is a lipocalin and is homologous to the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
8) the fragment or functional equivalent of 3), 4), 5), or 6), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
9) the fragment or functional equivalent of 3), 4), 5), or 6), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
10) the fragment or functional equivalent of 3), 4), 5), or 6), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
11) the fragment or functional equivalent of 3), 4), 5), or 6), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
12) the fragment or functional equivalent of 3), 4), 5), or 6), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
13) the polypeptide of any of 1) to 7), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
14) the fragment of any of 3), 8), 9), 10), 11), or 12), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
15) the polypeptide of any of 1) to 14), wherein the polypeptide contains amino acid substitution N92T, G114S, or both; or
16) the fragment of 14), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
17) a fusion polypeptide comprising the polypeptide according to any of 1) to 16); or
18) the of any of 1) to 17), further comprising a histidine tag; or
19) the polypeptide of 18), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72; and wherein
b) the nucleic acid molecule:
1) comprises a nucleic acid sequence encoding a polypeptide of any one of a1) to a19); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) hybridizes under high stringency conditions with a nucleic acid molecule of any of b1) to b3).

65: The method of claim 64, wherein said disease is selected from among vision disorders (e.g., nightblindness), immune system disorders (e.g., autoimmune disorders), inflammatory disorders, inflammatory bowel disease (IBD), ulcerative colitis (UC), Crohn's disease (CD), proctitis, cell proliferative disorders, cancer (e.g., breast cancer, cutaneous T-cell lymphoma, squamous cell carcinoma and/or basal cell carcinoma), microbial infections (e.g., viral, bacterial and fungal infections), emphysema, skin diseases (e.g. a Th1 skin disease such as psoriasis or hyperkeratotic dermatose; a Th2 skin disease such as atopic dermatitis, contact dermatitis, contact allergy to e.g., nickel or gold, cutaneous T-cell lymphoma, atopic eczema, acute eczema and/or chronic eczema), reproductive disorders (e.g., infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis and/or viral infections.

66: The method of claim 50, wherein said method of using a composition of matter comprises the method for identification of a compound that is effective in the treatment and/or diagnosis of a disease, comprising contacting a polypeptide or a nucleic acid molecule with one or more compounds suspected of possessing binding affinity for said polypeptide or nucleic acid molecule, and selecting a compound that binds specifically to said nucleic acid molecule or polypeptide, wherein
a) the polypeptide comprises:
1) an amino acid sequence comprising that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
2) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
3) a fragment of 1) or 2), wherein the fragment is a lipocalin or that has an antigenic determinant in common with the polypeptide according to 1) or 2); or
4) a functional equivalent of 1), 2), or 3); or
5) the fragment of 3), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO: 18, or amino acids 25-206 of SEQ ID NO:24; or
6) the fragment of 3), wherein the fragment is a lipocalin and consists of amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
7) the functional equivalent of 4), wherein the functional equivalent is a lipocalin and is homologous to the amino acid sequence recited in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
8) the fragment or functional equivalent of 3), 4), 5), or 6), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
9) the fragment or functional equivalent of 3), 4), 5), or 6), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
10) the fragment or functional equivalent of 3), 4), 5), or 6), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
11) the fragment or functional equivalent of 3), 4), 5), or 6), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
12) the fragment or functional equivalent of 3), 4), 5), or 6), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
13) the polypeptide of any of 1) to 7), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
14) the fragment of any of 3), 8), 9), 10), 11), or 12), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
15) the polypeptide of any of 1) to 14), wherein the polypeptide contains amino acid substitution N92T, G114S, or both; or
16) the fragment of 14), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
17) a fusion polypeptide comprising the polypeptide according to any of 1) to 16); or
18) the of any of 1) to 17), further comprising a histidine tag; or
19) the polypeptide of 18), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72; and wherein
b) the nucleic acid molecule:
1) comprises a nucleic acid sequence encoding a polypeptide of any one of a1) to a19); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:72; or
4) hybridizes under high stringency conditions with a nucleic acid molecule of any of b1) to b3).

67: The method of claim 66, wherein said disease is selected from among vision disorders (e.g., nightblindness), immune system disorders (e.g., autoimmune disorders), inflammatory disorders, inflammatory bowel disease (IBD), ulcerative colitis (UC), Crohn's disease (CD), proctitis, cell proliferative disorders, cancer (e.g., breast cancer, cutaneous T-cell lymphoma, squamous cell carcinoma and/or basal cell carcinoma), microbial infections (e.g., viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to e.g., nickel or gold, cutaneous T-cell lymphoma, atopic eczema, acute eczema and/or chronic eczema), reproductive disorders (e.g., infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis and/or viral infections.

68: The method of claim 50, wherein said method of using a composition of matter comprises the method for screening candidate compounds, comprising contacting a non-human transgenic animal with a candidate compound and determining the effect of the compound on the disease of the transgenic animal, wherein the transgenic animal has been transformed to express higher, lower, or absent levels of a polypeptide, wherein the polypeptide comprises:
a) the amino acid sequence comprising that recited in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
b) an amino acid sequence consisting of that recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24. SEQ ID NO:66, or SEQ ID NO:70; or
c) a fragment of a) or b), wherein the fragment is a lipocalin or that has an antigenic determinant in common with the polypeptide according to a) or b); or
d) a functional equivalent of a), b), or c); or
e) the fragment of c), wherein the fragment is a lipocalin and comprises amino acids 25-174 of SEQ ID NO:18, amino acids 26-180 of SEQ ID NO:18, amino acids 33-166 of SEQ ID NO:18, amino acids 41-189 of SEQ ID NO:18, or amino acids 25-206 of SEQ ID NO:24; or
f) the fragment of c), wherein the fragment is a lipocalin and consists of amino acids 25-174 of SEQ ID NO:18 amino acids 26-180 of SEQ ID NO:18, amino acids 33-166 of SEQ ID NO:18, amino acids 41-189 of SEQ ID NO:18, or amino acids 25-206 of SEQ ID NO:24; or
g) the functional equivalent of e), wherein the functional equivalent is a lipocalin and is homologous to the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
h) the fragment or functional equivalent of c), d), e), or f), wherein the fragment or functional equivalent has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
i) the fragment or functional equivalent of c), d), e), or f), wherein the fragment or functional equivalent has greater than 85% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
j) the fragment or functional equivalent of c), d), e), or f), wherein the fragment or functional equivalent has greater than 90% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 8, SEQ ID NO:20, SEQ ID NO:22, SEQ ID NO:24 or SEQ ID NO:66, or with an active fragment thereof; or
k) the fragment or functional equivalent of c), d), e), or f), wherein the fragment or functional equivalent has greater than 95% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
l) the fragment or functional equivalent of c), d), e), or f), wherein the fragment or functional equivalent has greater than 98% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:66, or with an active fragment thereof; or
m) the polypeptide of any of a) to g), wherein the polypeptide exhibits significant structural homology with a polypeptide having the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66 or SEQ ID NO:70; or
n) the fragment of any of c), h), i), j), k), or l), wherein the fragment has an antigenic determinant in common with the polypeptide of 1), and wherein the fragment consists of 7 or more amino acid residues from the amino acid sequence recited in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, or SEQ ID NO:70; or
o) the polypeptide of any of a) to n), wherein the polypeptide contains amino acid substitution N92T, G114S, or both; or
p) the fragment of n), wherein the fragment comprises the amino acid sequence of SEQ ID NO:36, SEQ ID NO:38, SEQ ID NO:40, SEQ ID NO:42, SEQ ID NO:44, SEQ ID NO:46, SEQ ID NO:56, SEQ ID NO:58, or SEQ ID NO:60; or
q) a fusion polypeptide comprising the polypeptide according to any of a) to p); or
r) the of any of a) to p), further comprising a histidine tag; or
s) the polypeptide of r), wherein the polypeptide comprises the amino acid sequence of SEQ ID NO:28, SEQ ID NO:30, SEQ ID NO:32, SEQ ID NO:34, SEQ ID NO:48, SEQ ID NO:50, SEQ ID NO:52, SEQ ID NO:54, SEQ ID NO:62, SEQ ID NO:64, SEQ ID NO:68, or SEQ ID NO:72.

69: The method of claim 68, wherein said disease is selected from among vision disorders (e.g., nightblindness), immune system disorders (e.g., autoimmune disorders), inflammatory disorders, inflammatory bowel disease (IBD), ulcerative colitis (UC), Crohn's disease (CD), proctitis, cell proliferative disorders, cancer (e.g., breast cancer, cutaneous T-cell lymphoma, squamous cell carcinoma and/or basal cell carcinoma), microbial infections (e.g., viral, bacterial and fungal infections), emphysema, skin diseases (e.g., a Th1 skin disease such as psoriasis or hyperkeratotic dermatose; a Th2 skin disease such as atopic dermatitis, contact dermatitis, contact allergy to e.g., nickel or gold, cutaneous T-cell lymphoma, atopic eczema, acute eczema and/or chronic eczema), reproductive disorders (e.g., infertility, in particular male infertility), renal dysfunction, myocardial infarction, arthritis, multiple sclerosis, gross cystic breast disease, regulation of nervous system development, Diabetes type 1, Hashimoto's disease, Grave's disease (thyroiditis), rheumatoid arthritis, proliferative and crescentic forms of glomerulonephritis, posterior uveitis, wound healing, and/or sarcoidosis, pityriasis rubra and/or porokeratosis, allergies such as allergic rhinitis, asthma, lichen rubber planus, chronic sinusitis, Sezary syndrome, actinic keratosis, hepatitis C, ulcerative colitis, membranous glomerulonephritis and/or viral infections.

70: A method of selecting biologically active compounds comprising:
(i) contacting a candidate compound with recombinant host cells expressing an INSP181 polypeptide; and
(ii) selecting compounds that bind said INSP181 polypeptide at the surface of said cells and/or that modulate the activity of the INSP181 polypeptide.

71: An isolated polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:66, SEQ ID NO:70.


SEQUENCE LISTING (69 sequences)

SEQ ID NO: 1 - DNA, 111 bp, Homo sapiens
atggccctggagaaaggcccgctcctgctgctggcccttggcctgggcctggcgggtgcccagaaggctctggaagaggtaccggtacagccgggcttcaatgcgcagaag

SEQ ID NO: 2 - protein, 37 aa, Homo sapiens
MALEKGPLLLLALGLGLAGAQKALEEVPVQPGFNAQK

SEQ ID NO: 3 - DNA, 137 bp, Homo sapiens
gtggaggggcgctggctcaccctgcagctggcagccaaccacgcagacctggtctccccggccgaccccctgaggctcgctctccactccatccggaccagggacggcggggacgtggacttcgtgctgttctggaa

SEQ ID NO: 4 - protein, 46 aa, Homo sapiens [sequence omitted]

SEQ ID NO: 5 - DNA, 74 bp, Homo sapiens
gggagaaggggtgtgtaaagaaacaaacatcaccgtccatccaacccagttgcaaggccagtaccaaggctcat

SEQ ID NO: 6 - protein, 25 aa, Homo sapiens
GEGVCKETNITVHPTQLQGQYQGSF

SEQ ID NO: 7 - DNA, 74 bp, Homo sapiens; identical to SEQ ID NO: 5

SEQ ID NO: 8 - protein, 25 aa, Homo sapiens
GEGVCKETNITVHPTQLQGQYQGSW

SEQ ID NO: 9 - DNA, 183 bp, Homo sapiens
ggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctgg

SEQ ID NO: 10 - protein, 61 aa, Homo sapiens
HGGVQGLGDGGERHRASAGPGSPTVEGGSMHVCFVSTDYSNLILYVRFEDDEITNLWVLLA

SEQ ID NO: 11 - DNA, 108 bp, Homo sapiens
tcgagggcggcagcatgcacgtatgcttcgtcagcaccgactacagcaacctcattctttacgtgcgctttgaggatgatgagatcaccaacctgtgggtgctgctgg

SEQ ID NO: 12 - protein, 36 aa, Homo sapiens
EGGSMHVCFVSTDYSNLILYVRFEDDEITNLWVLLA

SEQ ID NO: 13 - DNA, 96 bp, Homo sapiens
cgagaagaatgctggaggaccccaaatggctgggaagatacttggagtacgtggagaaattccacctgcagaaagccccggtcttcaacatagatg

SEQ ID NO: 14 - protein, 32 aa, Homo sapiens
RRMLEDPKWLGRYLEYVEKFHLQKAPVFNIDG

SEQ ID NO: 15 - DNA, 20 bp, Homo sapiens
gcccatgtcccccaccctga

SEQ ID NO: 16 - protein, 5 aa, Homo sapiens
PCPPP

SEQ ID NO: 17 - DNA, 546 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctga

SEQ ID NO: 18 - protein, 181 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPP

SEQ ID NO: 19 - DNA, 51 bp, Homo sapiens
cagaaggctctggaagaggtaccggtacagccgggcttcaatgcgcagaag

SEQ ID NO: 20 - protein, 17 aa, Homo sapiens
QKALEEVPVQPGFNAQK

SEQ ID NO: 21 - DNA, 486 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctga

SEQ ID NO: 22 - protein, 161 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPP

SEQ ID NO: 23 - DNA, 618 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 24 - protein, 206 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 25 - DNA, 558 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 26 - protein, 186 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 27 - DNA, 561 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggctgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 28 - protein, 187 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPPHHHHHH

SEQ ID NO: 29 - DNA, 501 bp, Homo sapiens
cagaaggctctggaagaggtaccggtacagccgggcttcaatgcgcagaaggtggaggggcgctggctcaccctgcagctggcagccaaccacgcagacctggtctccccggctgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 30 - protein, 167 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPPHHHHHH

SEQ ID NO: 31 - DNA, 636 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 32 - protein, 212 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 33 - DNA, 576 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 34 - protein, 192 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 35 - DNA, 74 bp, Homo sapiens
gggagaaggggtgtgtaaagaaacaaccatcaccgtccatccaacccagttgcaaggccagtaccaaggctcat

SEQ ID NO: 36 - protein, 25 aa, Homo sapiens
GEGVCKETTITVHPTQLQGQYQGSF

SEQ ID NO: 37 - DNA, 74 bp, Homo sapiens; identical to SEQ ID NO: 35

SEQ ID NO: 38 - protein, 25 aa, Homo sapiens
GEGVCKETTITVHPTQLQGQYQGSW

SEQ ID NO: 39 - DNA, 546 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctga

SEQ ID NO: 40 - protein, 181 aa, Homo sapiens [sequence omitted]

SEQ ID NO: 41 - DNA, 485 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctg

SEQ ID NO: 42 - protein, 161 aa, Homo sapiens
QKALEEVPVQPGFNAQKVEGRWLTLQLAANHADLVSPADPLRLALHSIRTRDGGDVDFVLFWKGEGVCKETTITVHPTQLQGQYQGSFEGGSMHVCFVSTDYSNLILYVRFEDDEITNLWVLLARRMLEDPKWLGRYLEYVEKFHLQKAPVFNIDGPCPPP

SEQ ID NO: 43 - DNA, 618 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 44 - protein, 206 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETTITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 45 - DNA, 558 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 46 - protein, 186 aa, Homo sapiens
QKALEEVPVQPGFNAQKVEGRWLTLQLAANHADLVSPADPLRLALHSIRTRDGGDVDFVLFWKGEGVCKETTITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 47 - DNA, 561 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggctgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 48 - protein, 187 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETTITVHPTQLQGQYQGSFEGGSMHVCFVSTDYSNLILYVRFEDDEITNLWVLLARRMLEDPKWLGRYLEYVEKFHLQKAPVFNIDGPCPPPHHHHHH

SEQ ID NO: 49 - DNA, 501 bp, Homo sapiens
cagaaggctctggaagaggtaccggtacagccgggcttcaatgcgcagaaggtggaggggcgctggctcaccctgcagctggcagccaaccacgcagacctggtctccccggctgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 50 - protein, 167 aa, Homo sapiens
QKALEEVPVQPGFNAQKVEGRWLTLQLAANHADLVSPADPLRLALHSIRTRDGGDVDFVLFWKGEGVCKETTITVHPTQLQGQYQGSFEGGSMHVCFVSTDYSNLILYVRFEDDEITNLWVLLARRMLEDPKWLGRYLEYVEKFHLQKAPVFNIDGPCPPPHHHHHH

SEQ ID NO: 51 - DNA, 636 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 52 - protein, 212 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETTITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 53 - DNA, 576 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaccatcaccgtccatccaacccagttgcaaggccagtaccaaggctcatggcatgggggggtccagggcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 54 - protein, 192 aa, Homo sapiens
QKALEEVPVQPGFNAQKVEGRWLTLQLAANHADLVSPADPLRLALHSIRTRDGGDVDFVLFWKGEGVCKETTITVHPTQLQGQYQGSWHGGVQGLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 55 - DNA, 183 bp, Homo sapiens
ggcatgggggggtccagagcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctgg

SEQ ID NO: 56 - protein, 61 aa, Homo sapiens
HGGVQSLGDGGERHRASAGPGSPTVEGGSMHVCFVSTDYSNLILYVRFEDDEITNLWVLLA

SEQ ID NO: 57 - DNA, 618 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagagcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 58 - protein, 206 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSWHGGVQSLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 59 - DNA, 558 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagagcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccc

SEQ ID NO: 60 - protein, 186 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSWHGGVQSLGDGGERHRASAGPGSPTVEGGSMHVCFVSTDYSNLILYVRFEDDEITNLWVLLARRMLEDPKWLGRYLEYVEKFHLQKAPVFNIDGPCPPP

SEQ ID NO: 61 - DNA, 636 bp, Homo sapiens
atggccctggagaaaggcccgctcctgctgctggcccttggcctgggcctggcgggtgcccagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagagcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 62 - protein, 212 aa, Homo sapiens
MALEKGPLLLLALGLGLAGAQKALEEVPVQPGFNAQKVEGRWLTLQLAANHADLVSPADPLRLALHSIRTRDGGDVDFVLFWKGEGVCKETNITVHPTQLQGQYQGSWHGGVQSLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 63 - DNA, 576 bp, Homo sapiens
cagaaggctctggaagaggtaccggtacagccgggcttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcatggcatgggggggtccagagcctgggggacggaggagagaggcatcgtgcatcagctggcccggggtctccaacagtcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccccaccatcaccatcaccat

SEQ ID NO: 64 - protein, 192 aa, Homo sapiens
QKALEEVPVQPGFNAQKVEGRWLTLQLAANHADLVSPADPLRLALHSIRTRDGGDVDFVLFWKGEGVCKETNITVHPTQLQGQYQGSWHGGVQSLGDGGERHRASAGPGSPTVEGGSMHVCFVSTDYSNLILYVRFEDDEITNLWVLLARRMLEDPKWLGRYLEYVEKFHLQKAPVFNIDGPCPPPHHHHHH

SEQ ID NO: 65 - DNA, 137 bp, Homo sapiens
gtggaggggcgctggctcaccctgcagctggcagccaaccacgcagacctggtctccccggctgaccccctgaggctcgctctccactccatccggaccagggacggcggggacgtggacttcgtgctgttctggaa

SEQ ID NO: 66 - protein, 149 aa, Homo sapiens
FNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPP

SEQ ID NO: 67 - DNA, 450 bp, Homo sapiens
ttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctga

SEQ ID NO: 68 - protein, 155 aa, Homo sapiens
FNAQKVEGRWLTLQLAANHADLVSPADPLRLALHSIRTRDGGDVDFVLFWKGEGVCKETNITVHPTQLQGQYQGSFEGGSMHVCFVSTDYSNLILYVRFEDDEITNLWVLLARRMLEDPKWLGRYLEYVEKFHLQKAPVFNIDGPCPPPHHHHHH

SEQ ID NO: 69 - DNA, 468 bp, Homo sapiens
ttcaatgcgcagaaggtggaggggcgctggctcaccctgcagctggcagccaaccacgcagacctggtctccccggccgaccccctgaggctcgctctccactccatccggaccagggacggcggggacgtggacttcgtgctgttctggaagggagaaggggtgtgtaaagaaacaaacatcaccgtccatccaacccagttgcaaggccagtaccaaggctcattcgagggcggcagcatgcacgtatgcttcgtcagcaccgactacagcaacctcattctttacgtgcgctttgaggatgatgagatcaccaacctgtgggtgctgctggcgagaagaatgctggaggaccccaaatggctgggaagatacttggagtacgtggagaaattccacctgcagaaagccccggtcttcaacatagatggcccatgtcccccaccctgacaccatcaccatcaccat